(19) United States
(12) Patent Application Publication
(10) Pub. No.: US 2012/0102582 A1
(43) Pub. Date: Apr. 26, 2012

(54) MOUSE MODELS

(76) Inventors: Barton F. Haynes, Durham, NC (US); Laurent K. Verkoczy, Durham, NC (US)

(21) Appl. No.: 13/262,714

(22) PCT Filed: Apr. 5, 2010
(86) PCT No.: PCT/US2010/030011

§ 371 Date: Dec. 27, 2011

Related U.S. Application Data
(60) Provisional application No. 61/202,778, filed on Apr. 3, 2009.

(51) Int. Cl. A01K 67/027; C12N 5/20; C12Q 1/70; C07K 16/10

(52) U.S. Cl. 800/18; 530/387.3; 435/328; 435/5

(57) ABSTRACT

The present invention relates, in general, to animal models suitable for testing candidate immunogens and, in particular, to knock-in mice expressing heavy and light chains of membrane proximal external region (MPER) HIV-I broadly neutralizing antibodies and to methods of screening candidate immunogens using same.

This application claims priority from U.S. Provisional Application No. 61/202,778, filed Apr. 3, 2009, the entire content of which is incorporated herein by reference.

### TECHNICAL FIELD

The present invention relates, in general, to animal models suitable for testing candidate immunogens and, in particular, to knock-in mice expressing heavy and light chains of membrane proximal external region (MPER) HIV-1 broadly neutralizing antibodies and to methods of screening candidate immunogens using same.

### BACKGROUND

The first antibodies that are made in acute HIV-1 infection are against the CD4 binding site (Moore et al, J. Virol. 68(8) 5142 (1994)), the CCR5 co-receptor binding site (Choe et al, Cell 114(2):161-170 (2003)), and the V3 loop (Moore et al, J. Acquit. Immun. Def. Syn. 7(4):332 (1994)). However, these antibodies do not control HIV-1 and are easily escaped (Burton et al, Nature Immun. 5:233-236 (2004), Wei et al, Nature 422(6929):307-312 (2003)). Neutralizing antibodies against autologous virus develop fifty to sixty days after infection, but antibodies capable of neutralizing heterologous HIV-1 strains do not arise until after the first year of infection (Richman et al, Proc. Natl. Acad. Sci. USA 100(7):4144-4149 (2003), Wei et al, Nature 422(6929):307-312 (2003)).

The four epitopes on HIV-1 envelope to which rare broadly reactive neutralizing antibodies bind are the CD4 binding site (CD4BS) (nab (monoclonal antibody) IgG1b12) (Zwick et al, J. Virol. 77(10):5863-5876 (2003)), the membrane proximal external region (MPER) epitopes defined by human mabs 2F5 and 4E10 (Armbruster et al, J. Antimicrob. Chemother. 54:915-920 (2004), Stiegler and Katinger, J. Antimicrob. Chemother. 51:757-759 (2003), Zwick et al, Journal of Virology 79:1252-1261 (2005), Purtscher et al, AIDS 10:587 (1996)) (FIG. 1), and the mannan glycan epitope defined by human mab 2G12 (Scanlan et al, Adv. Exper. Med. Biol. 535:205-218 (2003)). These four rare human mabs are all unusual: two are IgG3 (2F5 and 4E10), one has a unique Ig dimer structure (2G12), one has a very hydrophobic CDR3 (2F5) (Ofek et al, J. Virol. 198:10724 (2004)), and, in all four, the CDR3 is unusually long (Burton et al, Nature Immunol. 5(3):233-236 (2004), Kunert et al, AIDS Res. Hum. Retroviruses 20(7):755-762 (2004), Zwick et al, J. Virol. 78(6):3155-3161 (2004), Cardoso et al, Immunity 22:163-172 (2005)). Of these, 2F5- and 4E10-like human mabs are quite rare. Acute HIV-1 patients do not make antibodies against the MPER or 2G12 epitopes, MPER can be defined as amino acids 652 to 683 of HIV envelope is (Cardoso et al, Immunity 22:163-173 (2005) (e.g., QQEKNEQELLELDKWASLWNWFDITNWLWYIK). CD4 binding site (BS) antibodies are commonly made early in HIV-1 infection, but these antibodies generally do not have the broad spectrum of neutralization shown by mab IgG1b12 (Burton et al, Nat. Immunol. 5(3):233-236 (2004)).

A number of epitopes of the HIV-1 envelope have been shown to cross-react with host tissues (Pinto et al, AIDS Res. Hum. Retrov. 10:823-828 (1994), Douvas et al, AIDS Res, Hum. Retrov. 10:253-262 (1994), Douvas et al, AIDS Res. Hum. Retrov. 12:1509-1517 (1996)), and autoimmune patients have been shown to make antibodies that cross-react with HIV proteins (Pinto et al, AIDS Res. Hum. Retrov. 10:823-828 (1994), Douvas et al, AIDS Res, Hum. Retrov.

10:253-262 (1994), Douvas et al, AIDS Res, Hum. Retrov, 12:1509-1517 (1996), Barthel et al, Semin. Arthr. Rheum. 23:1-7 (1993)). Similarly, induction of immune responses to self-epitopes has been suggested to be a cause of the autoimmune abnormalities and T cell depletion in AIDS (Douvas et al, AIDS Res. Hum. Retrov. 12:1509-1517 (1996), Ziegler et al, Clin. Immunol. Immunopath. 41:305-313 (1986)).

The present invention results from studies designed to directly examine the role of B cell tolerance in regulating MPER-specific B cells and to determine the mechanisms involved/B cell subsets affected. The knock-in mouse models described herein can be used to yield genetic information on the spectrum of heavy and light chains within the MPER-specific B cell repertoire capable of conferring autoreactivity and/or neutralization activity. The disclosed mouse models can also be used to facilitate examination of lead candidate immunogens in eliciting MPER bnAbs, regardless of whether tolerance is involved or not.

### SUMMARY OF THE INVENTION

In general, the present invention relates to animal models suitable for testing candidate immunogens. More specifically, the invention relates to knock-in mice expressing heavy and light chains of MPER HIV-1 broadly neutralizing antibodies. The invention further relates to methods of screening candidate immunogens using such mice.

Objects and advantages of the present invention will be clear from the description that follows.

### BRIEF DESCRIPTION OF THE DRAWINGS

FIG. 1. General approach for generating broadly neutralizing antibody (bnAb) knock-in (ki) mice.

FIG. 2. Demonstration that 2F5 V_{H} can be expressed in association with mouse IgM and IgG constant domains.

FIG. 3. Representative staining profile demonstrating that ˜80% of bone marrow B cells in 2F5 VH^{+/-} mice are deleted at an early stage in B cell development (i.e., the pre-B to immature B cell transition), when the 2F5 heavy chain first pairs/expresses with light chains on the B cell surface.

FIG. 4. Representative staining showing an accumulation of a B cells in the periphery with an immature, anergic (functionally inactive, non-Ab secreting)-like phenotype in 2F5 V_{H}^{+/-} mice.

FIG. 5. Demonstration of a subset of autoreactive+MPER epitope-reactive B cells within the 2F5 heavy chain-expressing repertoire of 2F5 V_{H} knock-in mice.

FIGS. 6A and 6B. Two strategies aimed at testing MPER lead candidate immunogens in 2F5 knock-in mice in the absence of negative selection pressure.

FIG. 7. Stringent counterselection of 2F5 heavy chain-expression B cells by central and peripheral mechanisms in 2F5 V_{H} knock-in mice.

FIGS. 8A-8D. The chimeric, recombinant 2F5 antibody (m2F5) is is functionally equivalent to the original human 2F5 mAb (h2F5) in vitro. (FIGS. 8A and 8B) Functional comparison of m2F5 with h2F5 by Surface Plasmon Resonance (SPR) analysis demonstrating that antigen binding specificity and lipid reactivity are preserved in the recombinant m2F5 antibody, (FIG. 8A) m2F5 bound to the HIV-1 gp41 MPER peptides gp41₆₅₂₋₆₇₁ (top) and gp41₆₅₆₋₆₈₃ bottom) which include the 2F5 binding epitope (ELDKWAS). The binding of m2F5 (solid line) to MPER peptides was comparable to those of h2F5 (dashed line). The CD4i gp120 mAb 17b (dotted line) was used as a negative control. (FIG. 8B) Lipid reactivity was retained in m2F5 antibody and binding to liposomes with either phosphatidylserine (dashed line) or cardiolipin (solid line) was observed with both h2F5 and m2F5 mAbs. (FIG. 8C) m2F5 and h2F5 antibodies react with comparable avidity and specificity to mouse nuclear antigens. (FIG. 8D) m2F5 and h2F5 antibodies have comparable reactivities to human nuclear antigens. In addition, m2F5 and h2F5 were tested in the TZM-bl HIV Env pseudovirus neutralization assay and both antibodies neutralized HIV-1 strains B.BG1168, B.SF162, B.QH0692, B.JRFL, and C.92UG0237, but not C.TV-1. IC50 neutralization titer values for m2F5 and h2F5 ranged from 0.35 to is 11.9 μg/ml, and from 0.06 to 1.8 μg/ml, respectively (Table 1).

FIG. 9. Targeted replacement of the J_{H} cluster with the 2F5 V_{H} gene. Genomic structure of the 2F5 V_{H} targeting construct, the endogenous Ig HC locus, the targeted allele after homologous recombination, and the targeted allele after Cre-mediated neo cassette deletion. The 2F5 V_{H} expression cassette is is comprised of a J558 H10 family V_{H} promoter (p), an H10 split leader sequence (L), and the pre-rearranged 2F5 V(D)J V_{H} segment (2F5 V_{H}). Exons are represented as closed boxes, the Igh intronic enhancer (E) is represented by a circle, the 2F5 V_{H}, neo, and CAG-DTA cassettes are represented by shaded boxes, and loxP sites are depicted as triangles. The indicated restriction fragment sizes are indicated for wild-type and targeted loci. 5′ and 3′ probes used to verify homologous recombination events at the 5′ and 3′ regions of the J_{H}-Eμ region, respectively, are shown as black bars. Also indicated are PCR primers to identify i) homologous recombinant clones (black arrows) ii) removal of the neo marker (gray arrows), and iii) germline-transmitted heterozygous (2F5 V_{H}^{+/-}) and homozygous (2F5 V_{H}^{+/+}) mice (colored arrows). B=Bwn HI, RV=EcoRV, N=Nde I.

FIGS. 10A and 10B. Flow cytometric analysis of B-cell development in the bone marrow of C57BL/6 (WT), 2F5 V_{+/-}, and 2F5 V_{+/+} mice. FIG. 10A: representative dot plot histograms, with numbers indicating the percentage of cells within total bone marrow B-cell populations (gated as singlet, live, lin⁻ cells; lin=Ter-119, Gr-1, CD11b, CD4, CD8). Bone marrow cells were isolated from 9-12 week old female mice. FIG. 10B: statistical analysis of bone marrow B-cell subset frequencies with each black, open, and gray circle representing an individual WT, 2F5 V_{+/-}, and 2F5 V_{HV}^{+/+} mouse, respectively; horizontal lines represent averages for each group. Significance values were determined by a two-tailed Student's test: *, p≦0.05; **, p≦0.001; ***, p≦0.0001; NS, not significant. Populations were defined as follows: pro/large pre-B (fractions A-C′; B220^{lo}CD43⁺), small pre-B (fraction D; B220^{lo}CD43⁻), immature B (B220^{int/lo}IgM^{lo}IgD⁻), transitional, T1+T2 (B220^{int}IgM^{int/hi}IgD^{lo}), and bone marrow mature (B220^{hi}IgM^{int}IgD^{hi}).

FIGS. 11A and 11B. Flow cytometric analysis of WT and 2F5 V_{H} transgenic HCs expressed on the surface of 2F5 B-cells. (FIG. 11A) Representative contour histograms of splenic B-cell populations from WT IgH^{b}/WT IgH^{a} and 2F5 V_{H} IgH^{b}/WT IgH^{a} F1 mice, with numbers indicating the percentage of IgM^{a+} and IgM^{b+} cells (bearing endogenous and 2F5 V_{H} transgene-bearing HCs, respectively) within the singlet, live, total B (B220CD19⁺) cell gate. (FIG. 11B) Frequencies of IgM^{a} positive cells within IgM⁺ bone marrow and splenic fractions shown for five and six mice/F1 group, respectively.

FIGS. 12A-12C. Flow cytometric analysis of splenic B-cell development in C57BL/6 (WT), 2F5 V_{H}^{+/-}, and 2F5 V_{H}^{+/+} mice. Representative dot plot histograms using the Allman classification scheme (Allman et al, J. Immunol. 167:6834-6840 (2001)), with numbers in top panels showing the number of total B-cells (B220⁺) that were CD93 (AA4.1)⁺ transitional or CD93⁻ (mature+marginal zone, MZ) B-cells (FIG. 12A), and further gated to allow for enumeration of transitional T1-T3 populations within the B220⁺CD93⁺ fraction (FIG. 12B) or enumeration of MZ and mature B-cell subsets within the B220⁺CD93⁻ fraction (FIG. 12C). Mean Fluorescence Intensities for staining of WT, 2F5 V_{H}^{+/-}, and 2F5 V_{H}^{+/+} mature B cell subsets with PE-labeled anti-IgM were 756, 506, and 342, respectively.

FIGS. 13A and 13B. Total or autoantigen-specific serum Ig levels, and gp41 MPER-specific serum Ig or B-cell reactivity in WT, 2F5 V_{H}^{+/-}, and 2F5 V_{H}^{+/+} mice. (FIG. 13A) Total IgM and IgG serum antibody levels. Each dot represents an individual mouse; horizontal lines represent mean serum antibody levels. Significance values were determined by a two-tailed Student's test: *, p<0.05; **, p<0.001; p<0.0001; NS, not significant. (FIG. 13B) Total serum Ig reactivity against plate-bound gp41 MPER 2F5 nominal epitope peptide, anti-nuclear antigens, and cardiolipin. For gp41 MPER, ANA, and cardiolipin reactivity assays, MRL/lpr serum was used as a positive control. Each dot represents an individual mouse; horizontal lines represent mean serum antibody levels.

FIGS. 14A-14C, Pairing of the chimeric 2F5 heavy chain with arbitrary endogenous mouse LCs can make functional antibodies that bind equally well to self antigens as those paired with the 2F5 LC. (FIGS. 14A and 14B) Recombinant antibodies MK-1, MK-4, MK-5, and MK-6 (made from pairing the chimeric 2F5 heavy chain with endogenous mouse LCs) lack reactivity to MPER, but ¾ retain reactivity with cardiolipin comparable to that of m2F5 (the m2F5HC+m2F5 LC recombinant antibody). (FIG. 14A) Recombinant antibodies were purified from supernatants and assayed by standard ELISA at 100 μg/ml for binding cardiolipin or the nominal epitope peptide of 2F5, gp41₆₅₂₋₆₇₁ MPER as previously described (Haynes et al, Science 308:1906-1908 (2005), Alam et al, J. Immunol 178:4424-s 4435 (2007)). m2F5 was used as a positive control for both cardiolipin and gp41 MPER binding. (FIG. 14B) ELISA analysis of cardiolipin reactivity of MK-1, MK-4, MK-5, and MK-6 (performed as in FIG. 14A), graphically represented over a full concentration range. P3=P3X63/Ag8 negative control paraprotein. (FIG. 14C) SPR binding analysis of cardiolipin (CL) reactivity in MK-1, MK-4, MK-5, and MK-6. SPR analysis and preparations of CL, phosphatidylcholine (PC) and PC:CL (3:1) liposomes were performed as previously described (Alam et al, J. Immunol. 178:4424-4435 (2007)). These results show that lipid binding reactivities of the recombinant antibodies MK-1, MK-4, and MK-6 are comparable to that of the control m2F5 HC+m2F5 LC antibody. Both m2F5 positive control and MK1-6 mAbs bound to cardiolipin but not to phosphatidylcholine (PC) liposomes, All antibodies were injected at 50 μg/mL at 30 μL/min.

FIGS. 15A-15C. Confirmation of targeted insertion of 2F5 V_{H} into the mouse Igh locus, (FIG. 15A) Representative Southern blot analysis of genomic DNA from parental (lane1) and four recombinant ES cell clones with targeted 2F5 V_{H} (lanes 2-5). Mutant (mt) and wild type (wt) bands were revealed in three ways: probing Nde I-digested DNA with a PCR product 5′ of the J_{H}-Eμ region (5′ probe; top panel) and by probing Bam HI-digested DNA with a PCR product 3′ of the J_{H}-Eμ region (3′ probe; middle panel) or with a neo-specific probe (lower panel). (FIG. 15B) Representative PCR analysis of neo-deleted, 2F5 V_{H}^{+/-} ES cell-derived offspring harboring germline transmission of the 2F5 V_{H} insertion. Shown are gel-fractionated PCR products amplified from germline-transmitted offspring tail DNA of an F1 heterozygous crossing, revealing a WT, heterozygous (2F5 V_{H}^{+/-}) and homozygous (2F5 V_{H}^{+/+}) mouse; expected WT allele-specific and targeted allele-specific amplicons are ˜0.4 kb and 0.5 kb, respectively. (FIG. 15C) PCR amplification of IgM and IgG transcripts in C57BL/6 (WT) and 2F5 V_{H} B-cells. PCR products representing IgM (lanes 3,4) and IgG1 rearrangements (lanes 9,10) were detected in both C57BL/6 and 2F5 V_{H}^{+/-} cDNAs using a common (V_{H} J558) primer, whereas IgM (lanes 5, 6) or IgG1 (lanes 11, 12) rearrangements, only detectable in 2F5 V_{H}^{+/-} cDNA, were detected using a 2F5 V_{H}-specific primer.

FIG. 16. Comparison of B cell development in bone marrow of 2F5 V_{H} and 3H9 homozygous knock-in mice. Shown are representative flow cytometric histograms of CD93, CD23, and CD21 cell surface expression in B cell fractions within total BM B cell populations (gated as singlet, live, lin⁻ cells; lin=Ter-119, Gr-1, CD11b, CD4, CD8). BM cells were isolated from 9-12 week old female mice, and stained and analyzed as described for FIG. 10.

FIG. 17. Statistical analysis of splenic B-cell subset frequencies within the total (B220+) B-cell fractions of C57BL/6 (WT), 2F5 V_{H}^{+/-}, and 2F5 V_{H}^{+/+} mice. B cell subsets were fractionated using the Allman classification scheme (Allman et al, J. Immunol. 167:6834-6840 (2001)) as shown in FIG. 12, and the data is graphically represented in the same way as for FIG. 10.

FIGS. 18A and 18B. Representative staining profile demonstrating that a large fraction of 4E10 VH-expressing B cells in the bone marrow of 4E10 mice (like 2F5 V_{H}-expressing bone marrow B cells in 2F5 V_{H}^{+/+} mice), are deleted at an early stage in B cell development (i.e., the pre-B to immature B cell transition, when the 4E10 heavy chain first pairs/expresses with light chains on the B cell surface). BM cells were isolated, subjected to flow cytometry, and defined as described for FIG. 3. B cell populations with profound reductions in frequencies are highlighted in red.

FIGS. 19A and 19B. Representative staining profile demonstrating that 2F5 V_{L}-expressing B cells undergo normal development in the bone marrow of 2F5 knock-in mice. BM cells were isolated, subjected to flow cytometry, and defined as described for FIG. 3.

FIGS. 20A-20C. B cell-specific expression of the anti-apoptotic survival factor bcl2 rescues 2F5 V_{H}-expressing B cells and serum Igs from B cell tolerance in vivo, resulting in higher frequencies of MPER+ B cells. 2F5 V_{H} Eμ-bcl2 tg mice were generated by breeding Eμ-bcl2 transgenic mice with 2F5 V_{H} mice. (FIG. 20A) Representative staining profile demonstrating that the Eμ-bcl2 transgene rescues survival and development of B cells in 2F5 V_{H} spleen and bone marrow, Bone marrow cells (top panel) and splenocytes (bottom panel) were isolated from 8-12 wk old female mice and total live B cells were stained using combinations of the B cell-specific markers IgM, IgD, B220, and CD19. (FIG. 20B) Comparison of serum IgM, IgG3, IgG1, and IgG2b levels demonstrating that serum IgM and IgG3, normally suppressed in 2F5 V_{H} knock-in mice, is selectively rescued in 2F5 V_{H} Eμ-bcl2 tg mice (suggesting that the bcl2 transgene may also rescue certain IgM and IgG3-producing B cell populations from anergy i.e. functional inactivation). Analysis of total serum immunoglobulin levels from 8-12 wk old B6 (WT), 2F5 V_{Hl}^{+/+}, or 2F5 V_{H}^{+/+} Eμ-bcl2 tg female mice was measured using a standard Luminex assay. (FIG. 20C) Hybridoma analysis demonstrating that bcl2 transgene expression results in increased frequencies of IgM+ splenic B cells having MPER reactivity. Hybridomas were made from LPS-activated 2F5 V_{H}^{+/+} or 2F5 V_{H}^{+/+}×bcl2 tg splenic cultures, hybridoma supernatants were sub-cloned for 2 rounds at limiting dilution and then were screened by ELISA for Cardiolipin (CL) or MPER reactivity. Data shown represents the frequency of CL and/oe MPER (Sp62)-specific wells/total IgM+ wells.

### DETAILED DESCRIPTION OF THE INVENTION

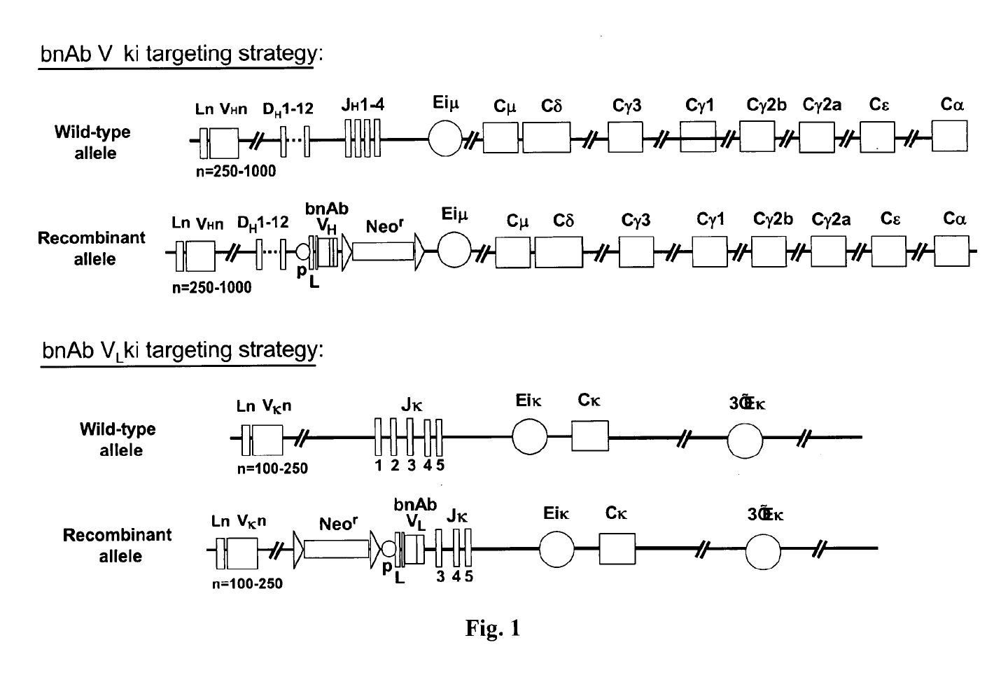

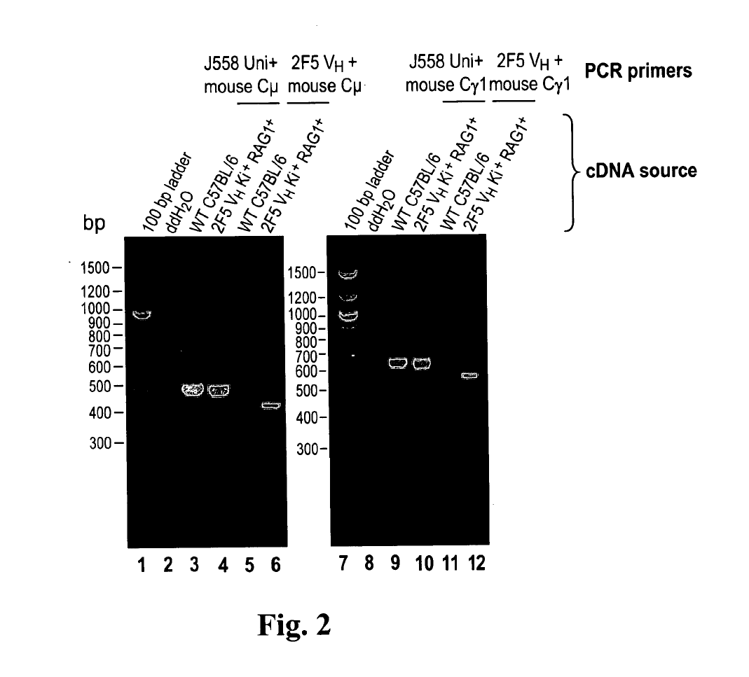

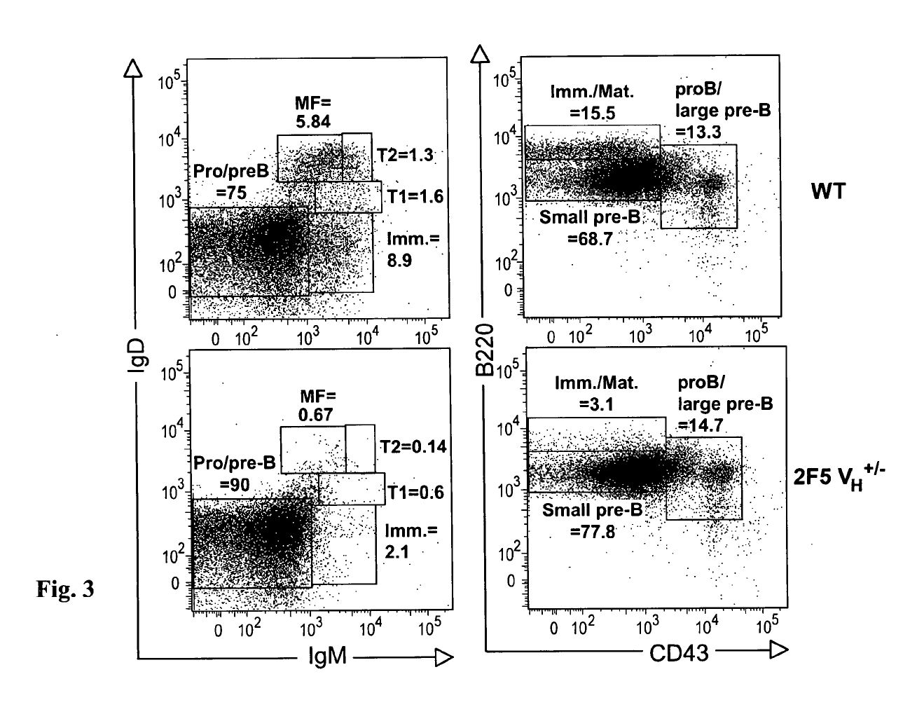

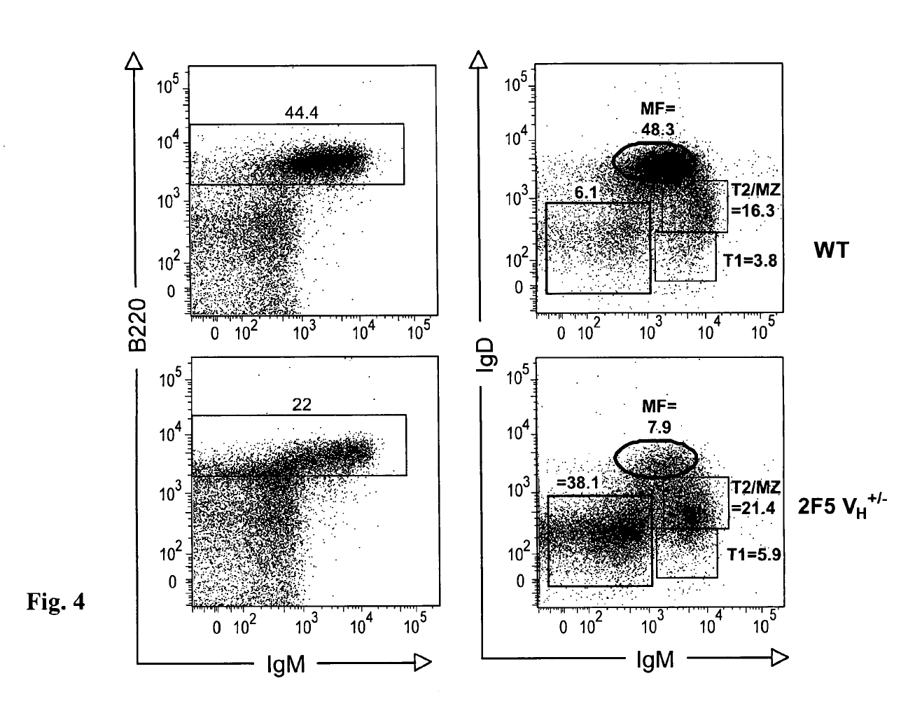

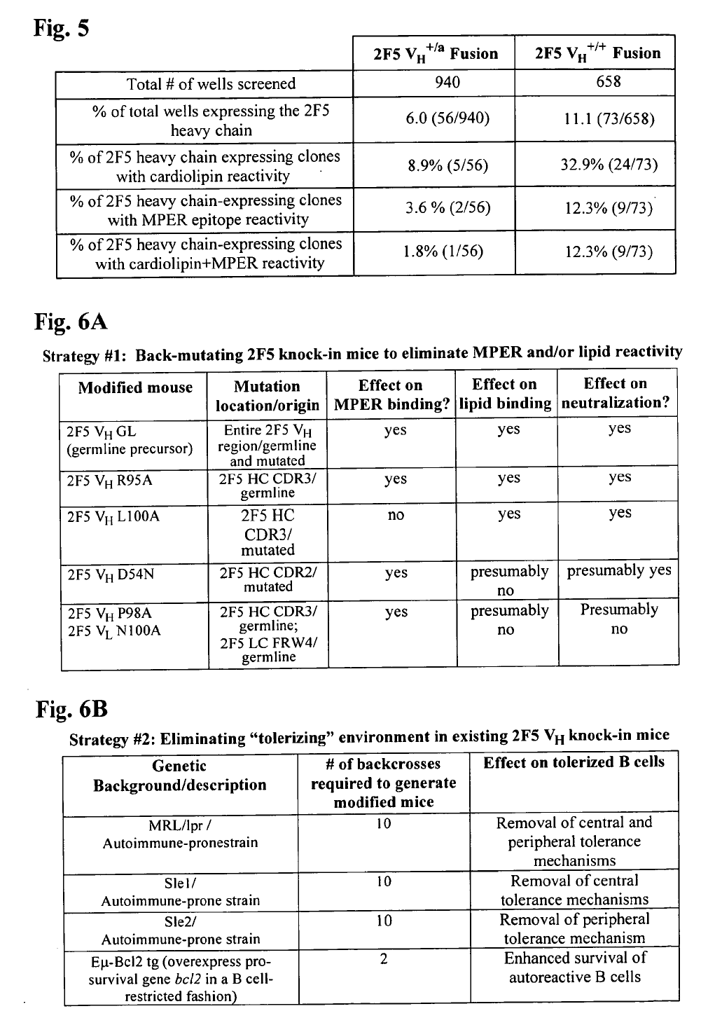

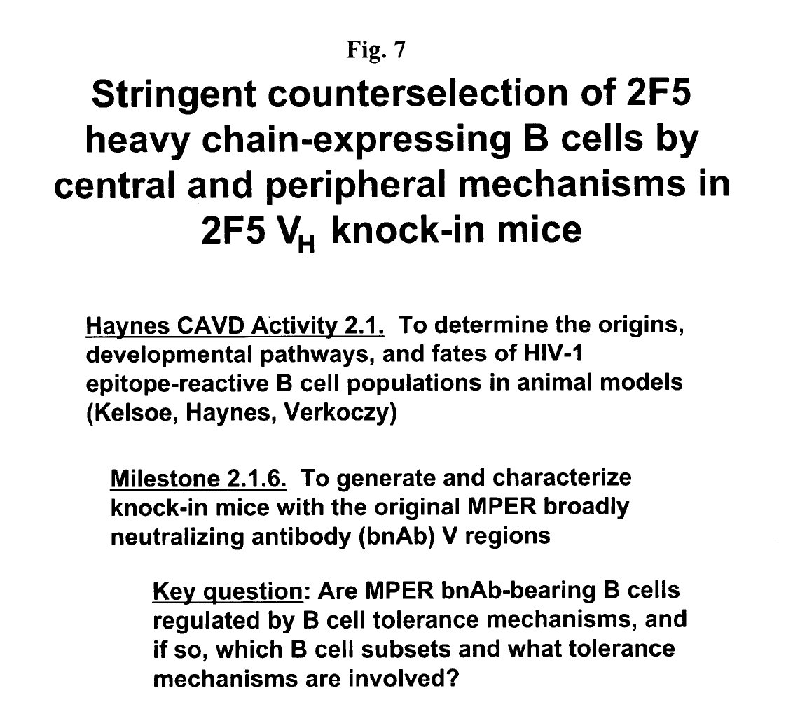

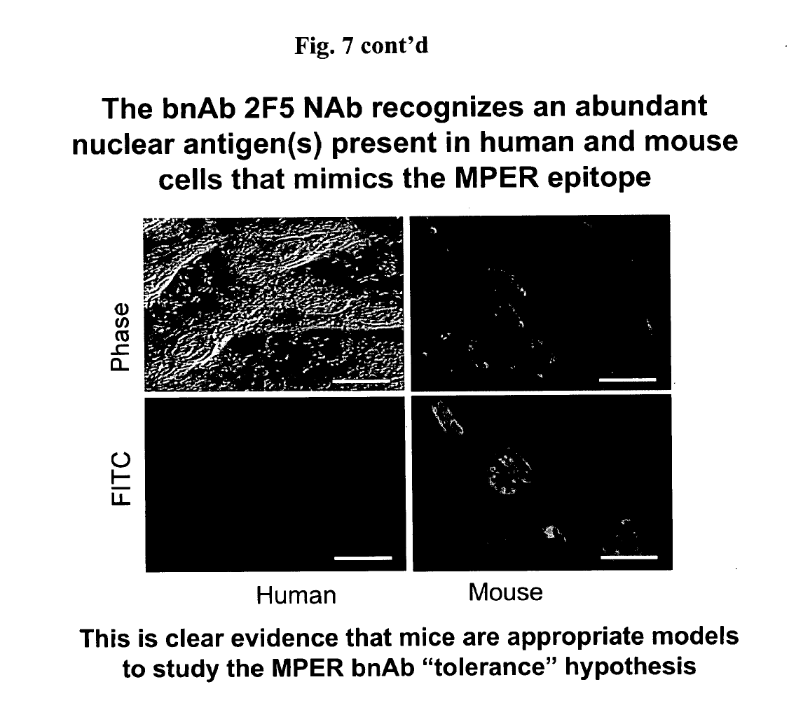

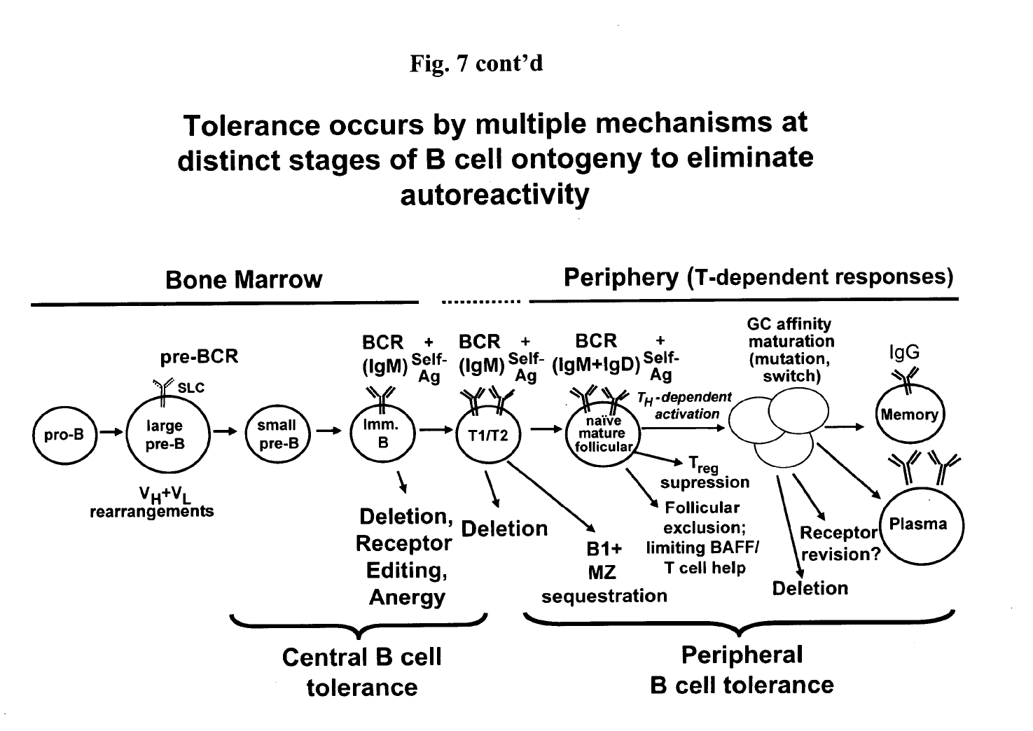

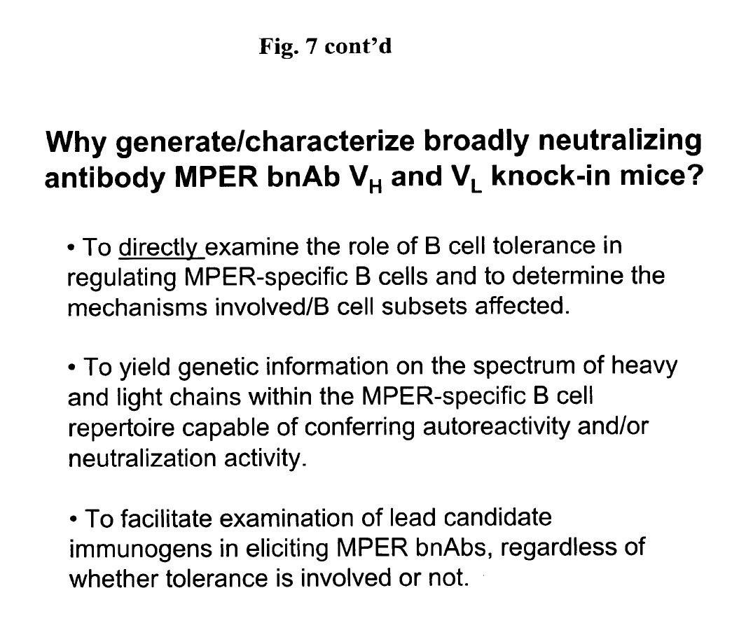

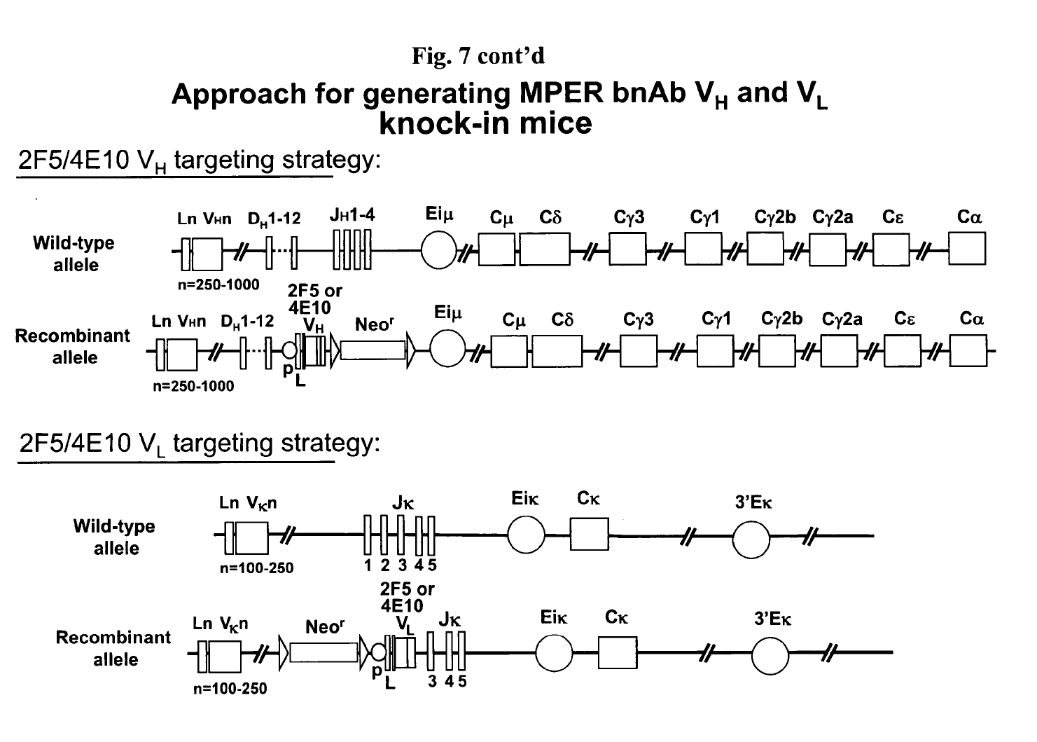

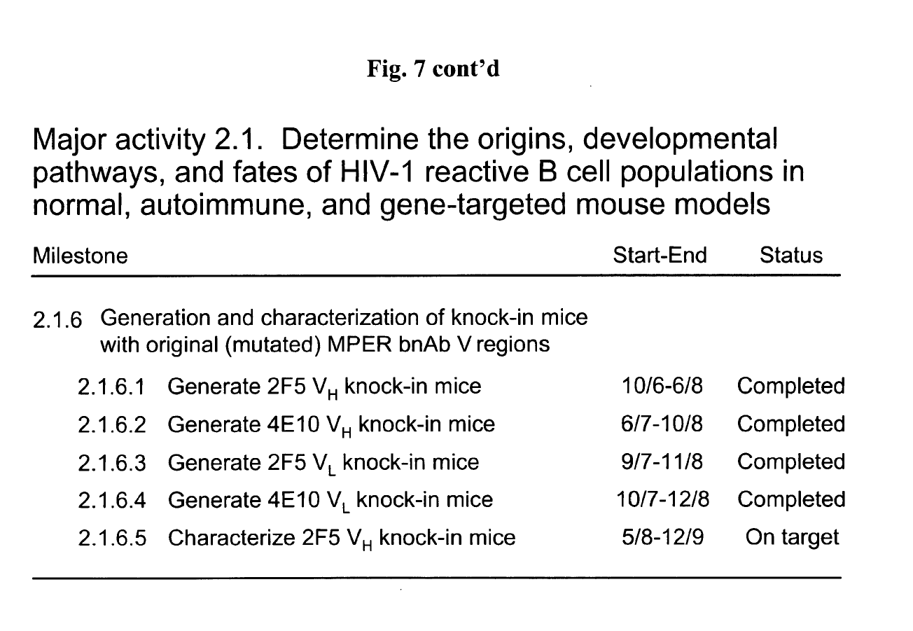

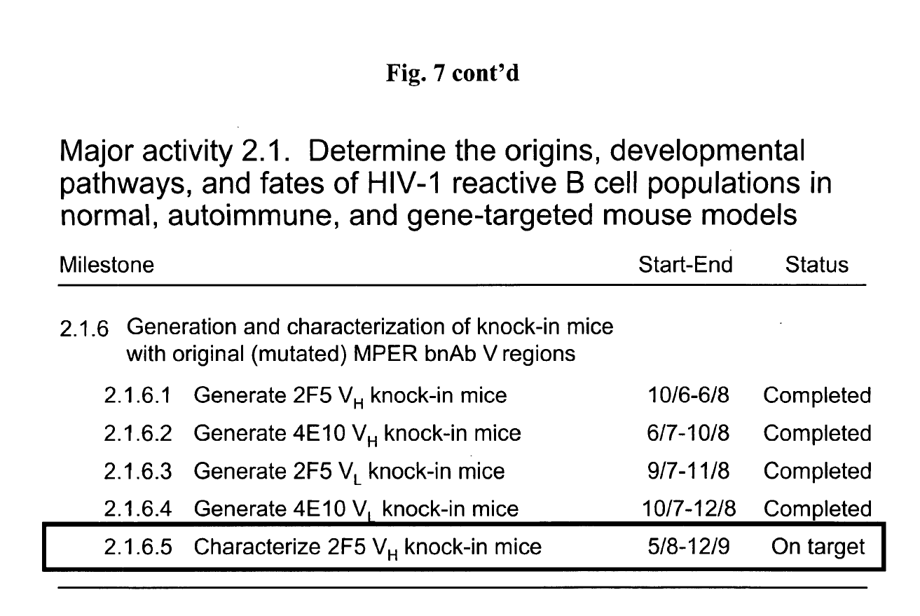

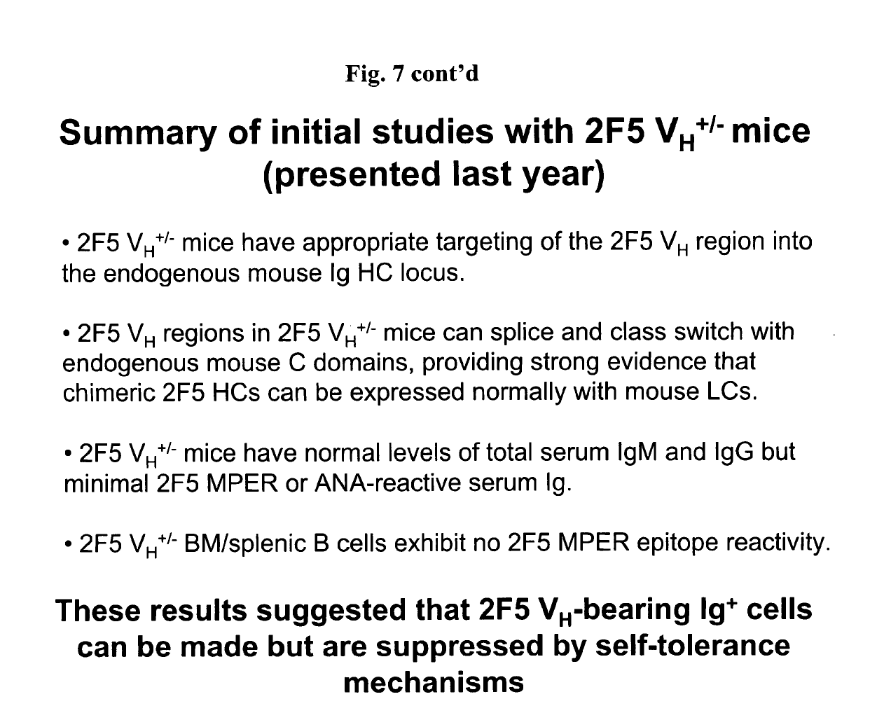

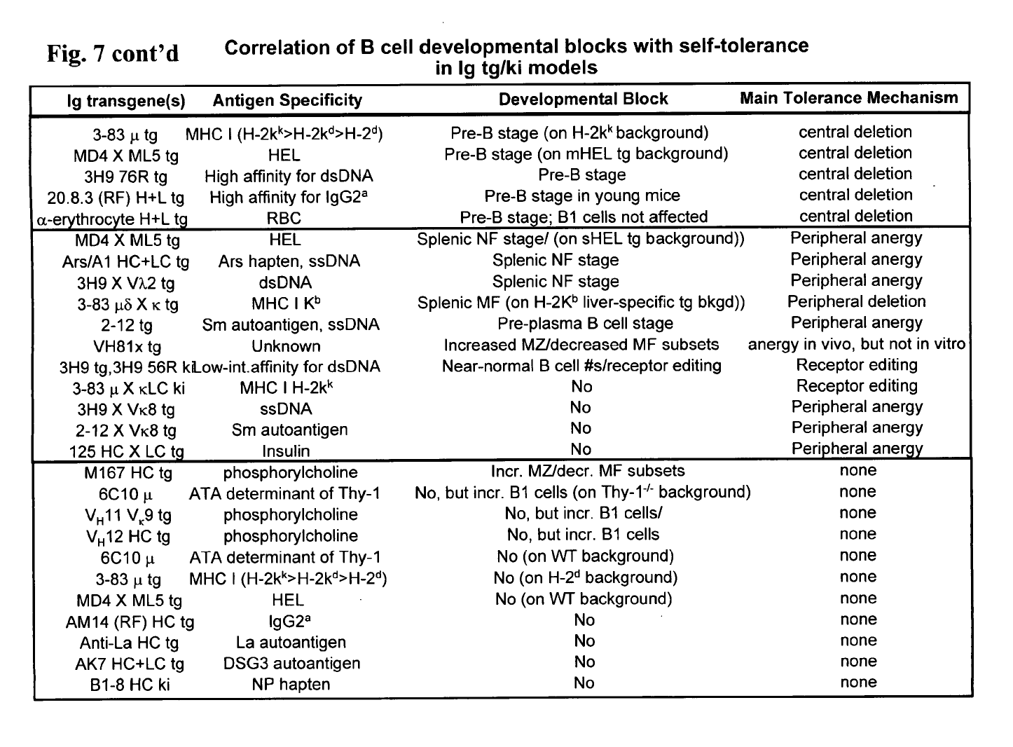

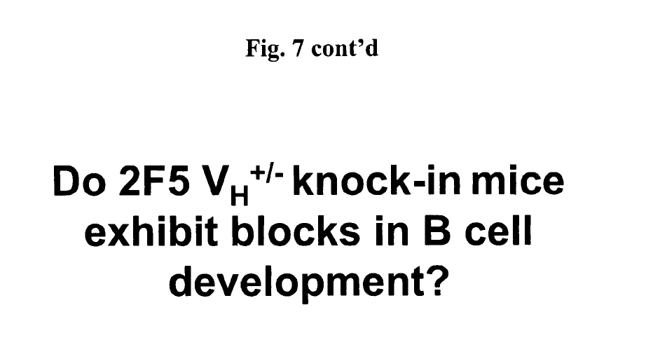

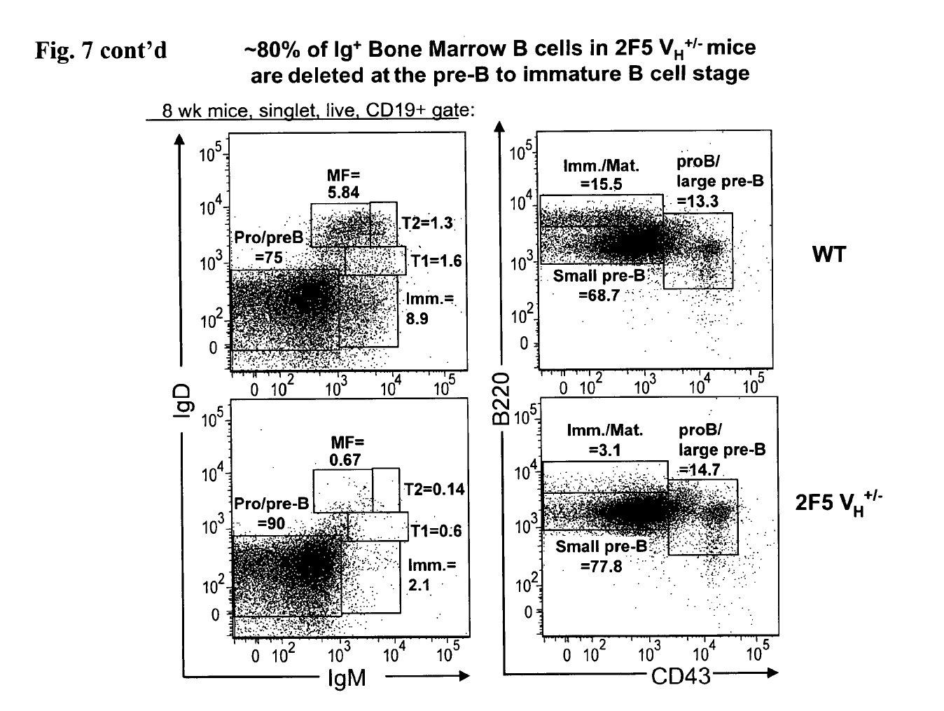

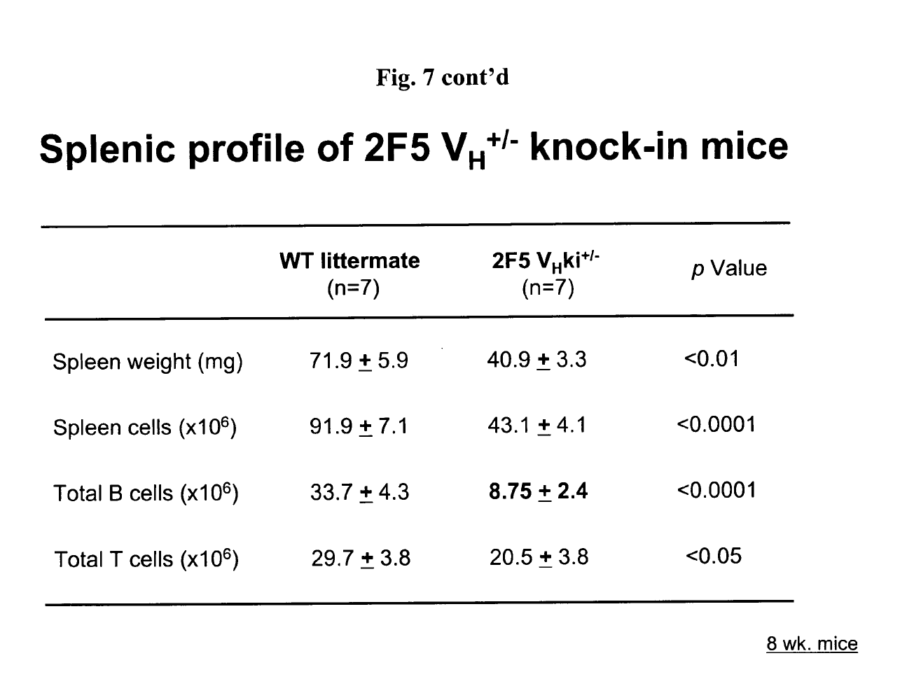

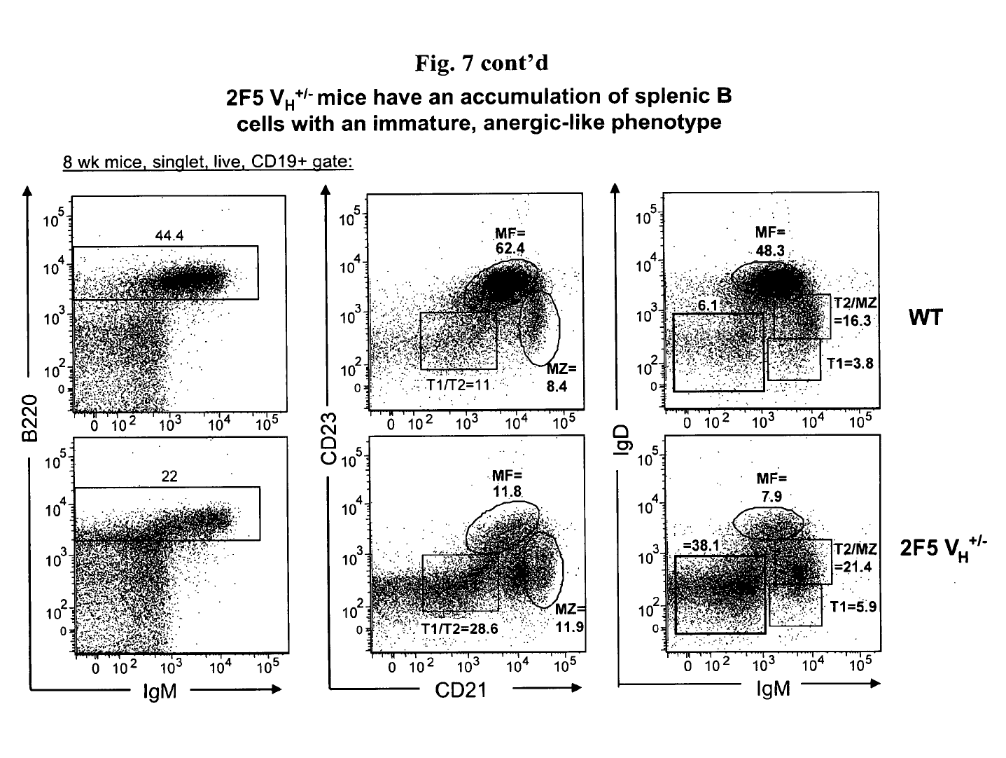

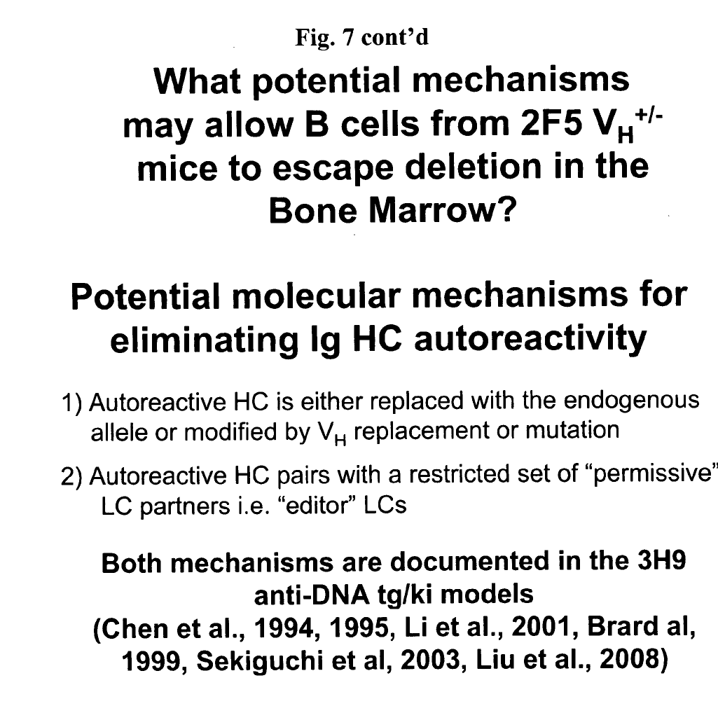

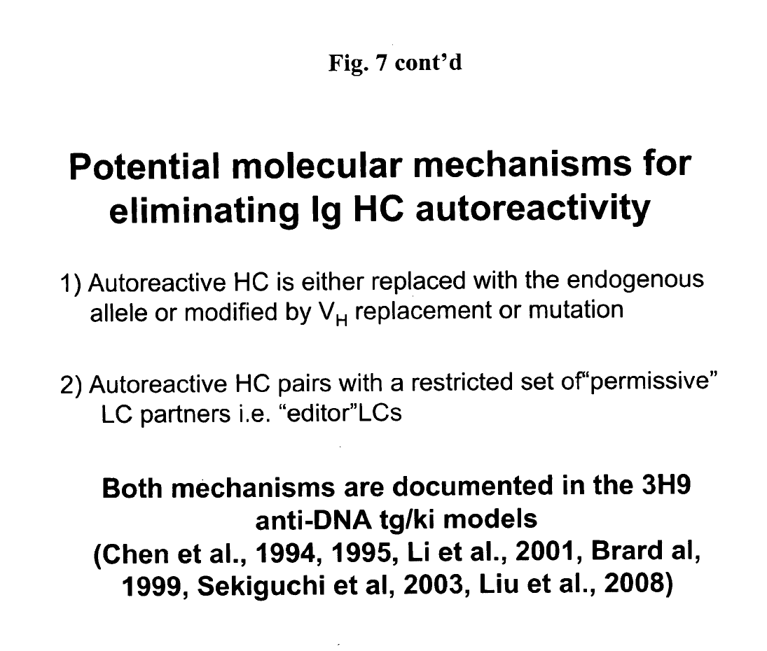

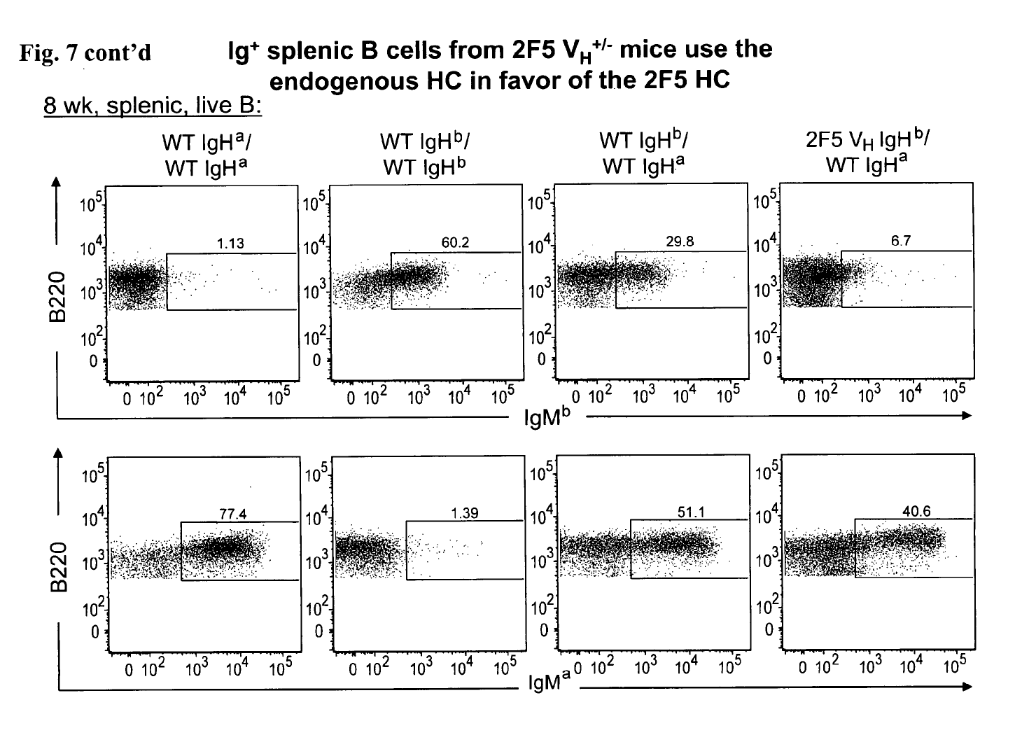

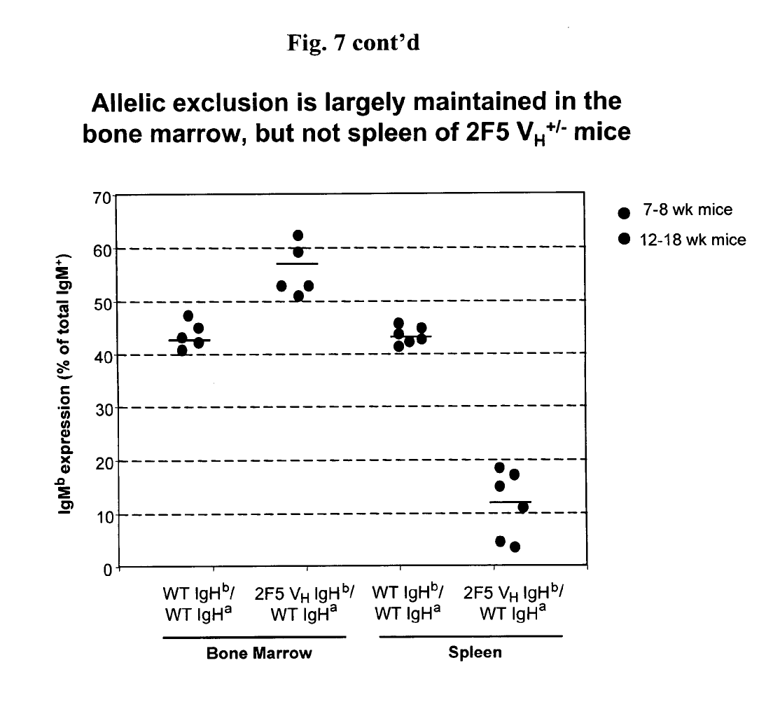

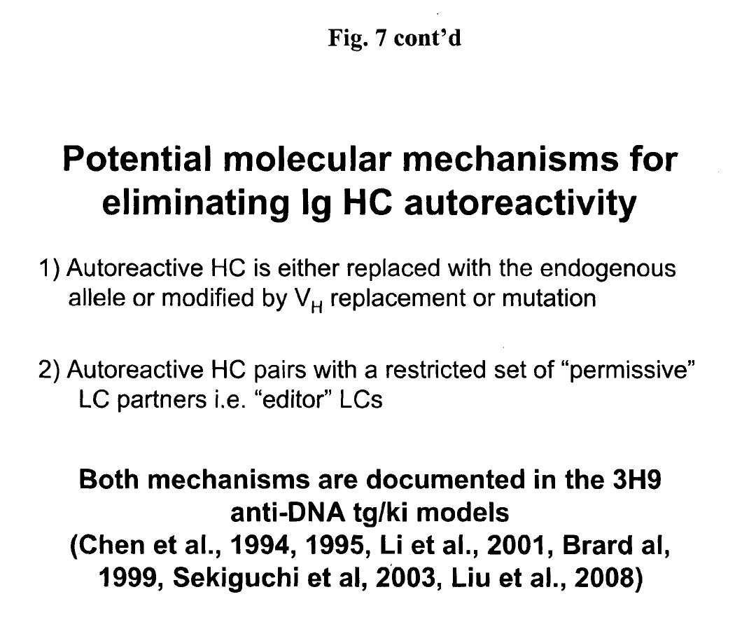

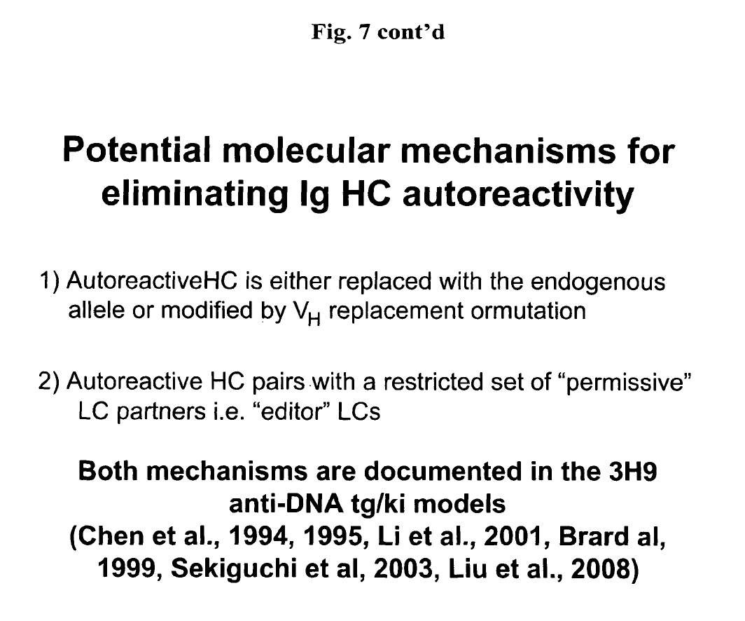

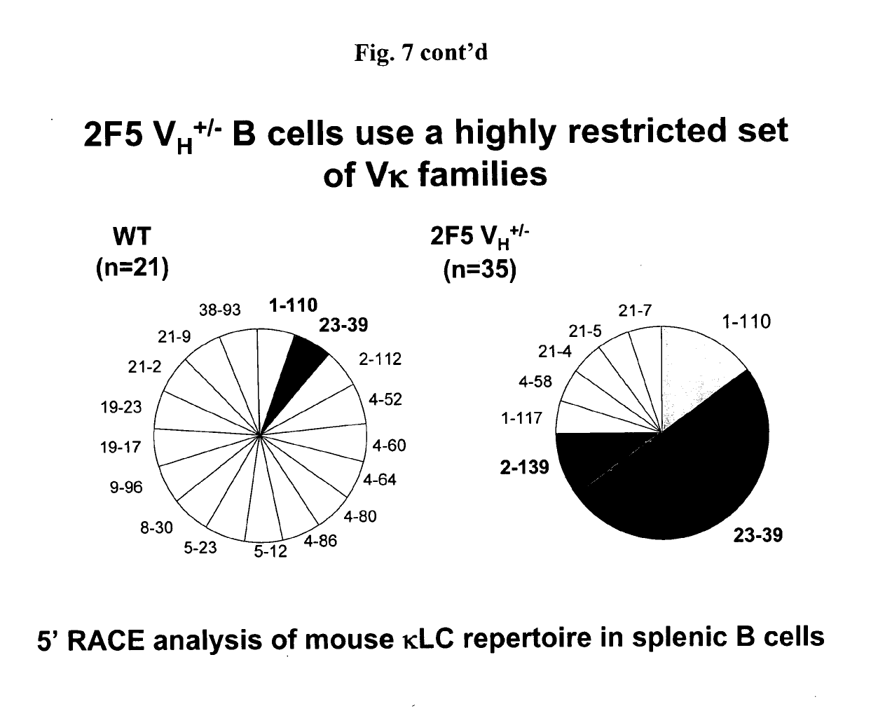

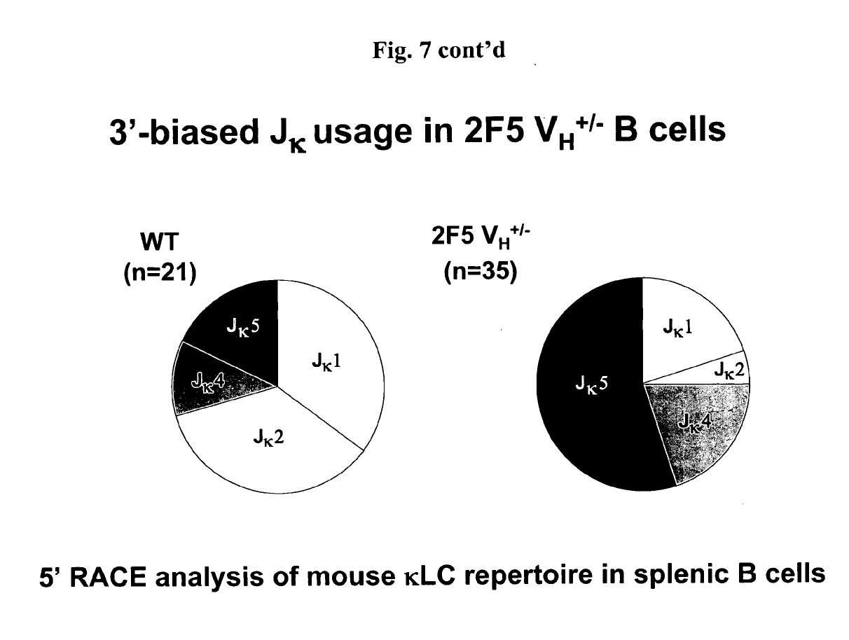

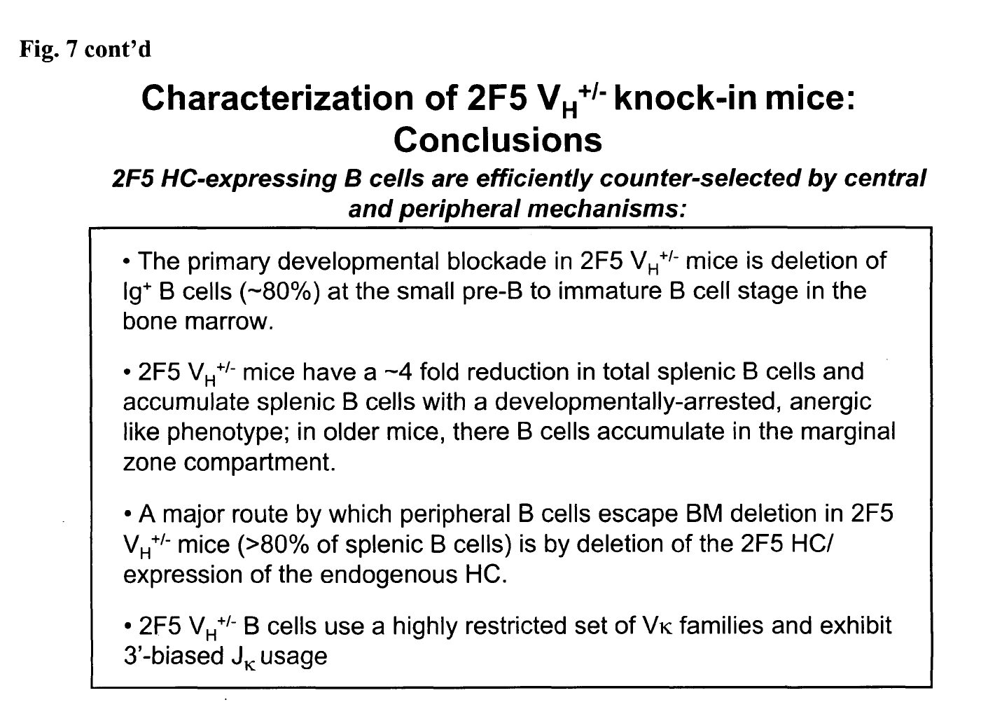

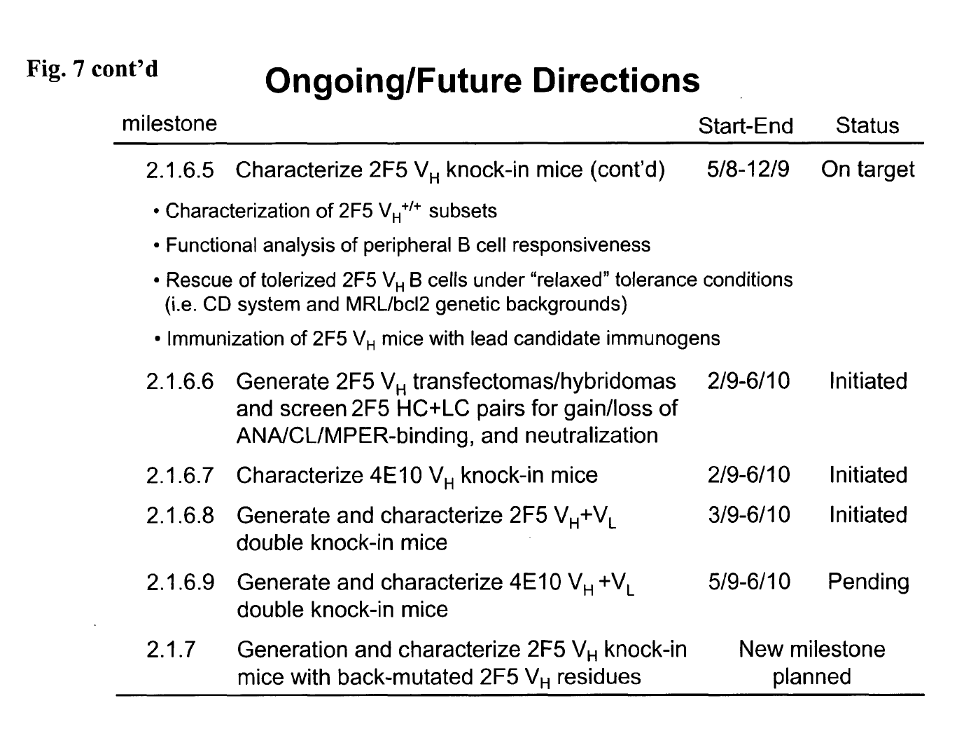

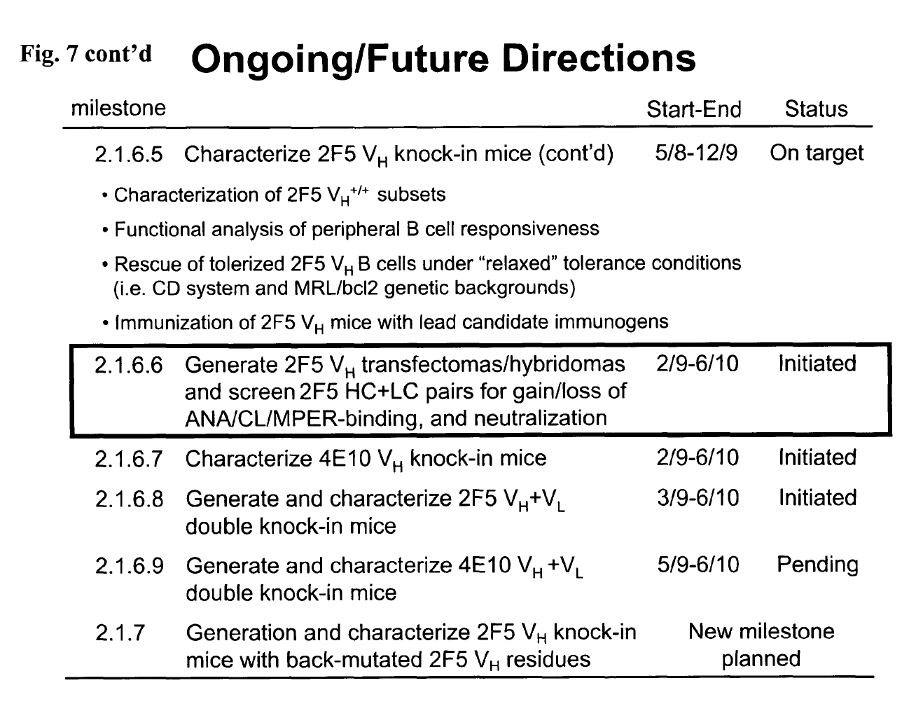

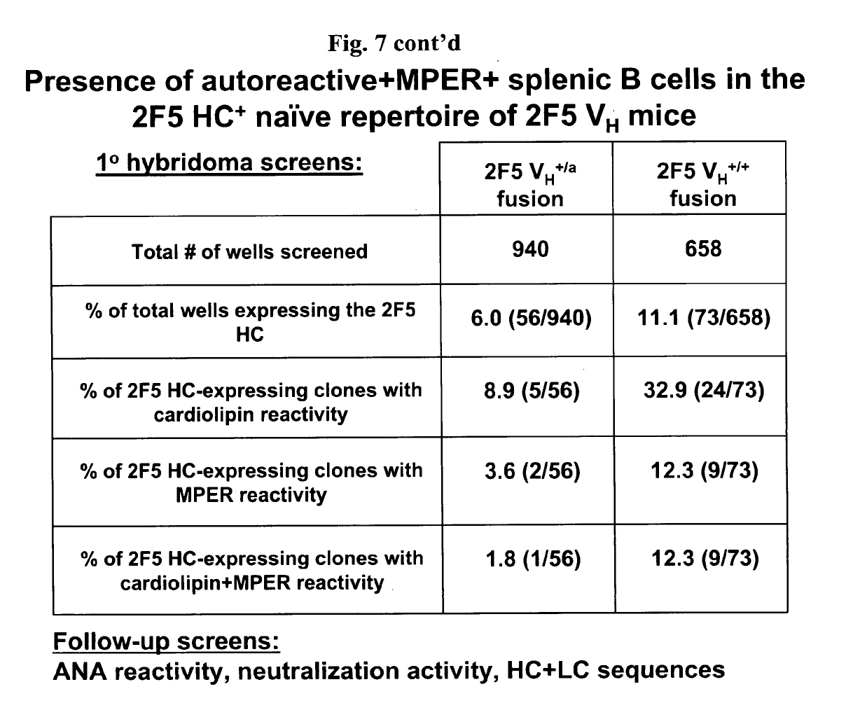

The present invention relates to knock-in animal (e.g., mouse) models expressing MPER HIV-1 broadly neutralizing antibodies and to methods of screening lead candidate immunogens using same. The invention results from the construction of a series of knock-in mouse lines expressing the heavy and light chains of two broadly neutralizing HIV-1 gp41 membrane proximal external region (MPER) antibodies (2F5 and 4E10) at their endogenous immunoglobulin loci (see FIG. 1 and Examples that follow). Characterization of one of these lines, the 2F5 V_{H} knock-in mouse (with site-directed expressing of the 2F5 heavy chain), has led to the critical observation that the 2F5 heavy chain can be appropriately expressed by B cells (FIG. 2), but that the majority of these 2F5-expressing B cells are eliminated early in B cell development, at the stage at which the heavy chain pairs with light chains (FIG. 3). Importantly, 2F5 V_{I-4} mice have a substantial subset of anergic-like 2F5-expressing B cells in the periphery that escape this initial counterselection step (FIG. 4), as well as a small subset of B cells capable of secreting 2F5-expressing antibodies that are autoreactive and MPER-reactive (FIG. 5). In this context, these mice represent an attractive model for directly testing how remaining 2F5-expressing B cells can be elicited, modulated, or expanded to secrete broadly neutralizing antibodies (bnAbs) by lead candidate immunogens.

Thus, in one embodiment, the present invention relates to a targeted transgenic mouse, the genome of which comprises a nucleic acid sequence encoding a heavy and/or a light chain variable region of a human HIV-1 broadly neutralizing antibody. In accordance with the invention, the nucleic acid sequence can be present in the genome operably linked to a promoter so that the nucleic acid sequence is expressed and the heavy and/or light chain variable region of the human HIV-1 broadly neutralizing antibody (e.g., 2F5 or 4E10) is produced. Advantageously, the nucleic acid sequence is present in the genome operably linked to an endogenous enhancer element

In a preferred embodiment, the nucleic acid sequence encoding the heavy chain variable region of the human HIV-1 broadly neutralizing antibody is operably liked to a J558 1110 family VH promoter (Love et al, Mol. Immunol. 37:29-39 (2000)). In another preferred embodiment, the nucleic acid sequence encoding the light chain variable region of the human HIV-1 broadly neutralizing antibody is operably liked to a VkOx-1 family Vkappa promoter (Sharpe et al, EMBO 10(8):2139-2145 (1991)).

The invention further relates to chimeric HIV-1 broadly neutralizing antibodies isolatable from the above-described targeted transgenic mouse, particularly, a mouse that contains in its genome both a nucleic acid sequence encoding a heavy chain variable region of a human HIV-1 broadly neutralizing antibody and a nucleic acid sequence encoding a light chain variable region of a human HIV-1 broadly neutralizing antibody. The invention also relates to hybridomas derived by fusing antibody-producing B cells of the above-described mouse with myeloma cells using, for example, standard techniques. The invention includes monoclonal antibodies produced by such hybridomas.

In yet another embodiment, the invention relates to a method of identifying a candidate agent capable of inducing the production of HIV-1 broadly neutralizing antibodies. The method can comprise: i) administering to the above-described mouse a test compound (e.g., a compound comprising a protein or peptide) under conditions such that antibodies can be produced or such that B cells can be induced to express antibodies, ii) obtaining an antibody-containing sample or an antibody expressing, B cell-containing sample from the mouse, and iii) assaying the sample for the presence or absence of antibodies specific for the HIV-1 membrane proximal external region (MPER), or MPER-specific B cells (e.g., using an ELISA, ELISPOT, Surface Plasmon Resonance, Luminex or flow cytometry-based assay). The presence of the MPER-specific antibodies or B cells in the sample, relative to a control sample (e.g., a non-treated mouse), indicates that the test compound is a candidate agent. The antibody-containing sample or the antibody expressing, B cell-containing sample can be a serum sample or a sample of a mucosal extract (e.g., a saliva, stool or vaginal wash sample). The B-cell containing sample can be a sample obtained from a systemic or mucosal immune tissue of the mouse. For example, the sample can be a bone marrow, spleen or peripheral blood lymphocyte sample. The sample can also be an enteric lymph node or Peyer's patch sample or a female reproductive tract sample or a lung sample. A candidate agent identified using the method of the invention can be assayed for HIV-1 neutralizing activity, for example, using TZM-bl assay (see, for example, Polonis et al, virol. 375(2) 315 (2008)).

The invention further relates to a method of identifying an agent capable of inducing the production of HIV-1 broadly neutralizing antibodies that comprises: i) administering to the above-described mouse a test compound (e.g., a proteinaceous compound) under conditions such that antibodies can be produced or such that B cells can be induced to express antibodies, ii) obtaining an antibody-containing sample or an antibody expressing, B cell-containing sample from the mouse, and iii) assaying the sample for HIV-1 neutralizing activity, relative to a control sample (e.g., from a non-treated mouse). As above step (iii) can be effected using, for example, a TZM-bl assay.

The data provided in the Examples below demonstrate that both heterozygous and homozygous versions of 2F5 V_{H} knock-in mice have been characterized. Both exhibit a major blockade in bone marrow B cell development at the pre-B to mature B cell transition, indicating that the developmental block cannot be due to a defect in transgene expression but, rather, must involve an active process involving encounter of the 2F5 HC with self-antigens. The ability of the 2F5 V_{H}-containing heavy chain to pair with multiple endogenous mouse light chain partners in vivo is demonstrated in Example 2 (see hybridoma analysis in Table 6), and strongly argues against the developmental block being due to improper association with mouse light chains. Despite diminished total numbers of mature splenic B cells with lower surface density, there are normal ratios of mature B cells in these mice, indicating that a small percentage of cells escape initial deletion in the bone marrow. Importantly, this shows that the developmental block is profound, but not complete, and residual cells have the potential to be rescued by a variety of strategies. In serum from heterozygous and homozygous 2F5 V_{H} knock-in mice, there are substantial levels of total serum Igs, but which lack reactivity to the MPER epitope or to human/murine self antigens. The ability of these mice to produce normal Ig levels shows that the lack of reactivity is not due to improper expression of the transgene but rather must be due to elimination of this reactivity by various tolerance mechanisms. In vitro, the 2F5 V_{H} insertion can produce chimeric human/mouse 2F5 antibodies that are functionally similar to the original human 2F5 antibody, including comparable reactivity to the MPER epitope+human/murine self-antigens, and the ability to neutralize HIV-1. This demonstrates that the lack of serum Ig reactivity in these mice is also not due to the chimeric nature of the 2F5 HC.

Furthermore, other knock-in models can be readily genetically manipulated to remove the above-described counterselection pressures (FIG. 6) and they also represent powerful in vivo models for dissecting which lead immungen candidates/immunization strategies can best shape the precursor bnAb B cell repertoire via somatic hypermutation or other B cell diversification processes, independent of B cell tolerance effects. Additionally, the knock-in approach described for generating HIV-1 bnAb specificities can be used analogously for expressing any bnAb specificity, thus making it possible to test candidate vaccine immunogens for their ability to elicit broadly neutralizing antibodies against any viral infectious agent. Finally, the potential impact of B cell immunomodulation in other viral infectious agent models can be studied using this knock-in approach, of particular relevance for viral infections with is similarities to HIV-1, for example, those exhibiting potential signatures of molecular mimicry or strong selection pressures which bias the V_{H} B cell repertoire, such as the V_{H}1-69 bias seen with bnAbs to Influenza and Hepatitis C.

Certain aspects of the invention can be described in greater detail in the non-limiting Examples that follows. (See also U.S. Provisional Application No. 61/166,625, filed Apr. 3, 2009 and U.S. Provisional Application No. 61/166,648, filed Apr. 3, 2009.)

### Example 1

FIG. 1 shows the general approach for generating broadly neutralizing antibody (bnAb) knock-in (ki) mice. bnAb 2F5/4E10 V_{H} and V_{L} ki mice were generated by the site-directed replacement of the endogenous mouse J_{H} or Jκ clusters (in red) with the original, mutated 2F5/4E10 V_{H}DJ_{H} or 2F5/4E10 VκJκ gene rearrangements (in blue), respectively. The 2F5/4E10 V_{H} cassettes are comprised of a J558 H10 family V_{H} promoter (p), the H10 split leader sequence (L), and the pre-rearranged 2F5/4E10 V(D)J V_{H} segments. The 2F5/4E10 V_{L} cassettes are comprised of a V_{κ}Ox1 promoter (p), the V_{κ}Ox1 split leader sequence (L), and the rearranged 2F5/4E10 VκJκ coding segments.

FIG. 2 demonstrates that 2F5 V_{H} can be expressed in association with mouse IgM and IgG constant domains. PCR products representing common V_{H}1 IgM (lanes 3,4) and IgG1 rearrangements (lanes 9,10) were detected in both C57BL/6 and 2F5 V_{H}ki cDNAs using the V_{H} J558 primer. In contrast, the expected 2F5 PCR product for either IgM (lanes 5, 6) or IgG1 (lanes 11, 12) was only detected in 2F5 V_{H}ki^{+/-} cDNA. These data show that 2F5 V_{H} is capable of being transcribed in chimeric animals both as IgM and IgG1, indicating appropriate expression and class switching of the human 2F5 V_{H}DJ_{H} rearrangement in mice, and capable of pairing with mouse light chains.

FIG. 3 provides a representative staining profile demonstrating that ˜80% of bone marrow B cells in 2F5 VH^{+/-} mice are deleted at an early stage in B cell development (i.e. the pre-B to immature B cell transition), when the 2F5 heavy chain first pairs/expresses with light chains on the B cell surface. BM cells were isolated from 8 wk Wild Type or 2F5 V_{H}^{+/-} and subjected to flow cytometry. B cell subsets from total B cell populations (singlet, live, lineage⁻, CD19⁺ and B220⁺ gated cells) were defined either using the Kalled subfractionation scheme (left panels, for distinguishing more mature B cell BM fractions) or the Hardy fractionation scheme (right panels, for distinguishing early B cell subsets).

FIG. 4 shows an accumulation of a B cells in the periphery with an immature, anergic (functionally inactive, non-Ab secreting)-like phenotype in 2F5 V_{H}^{+/-} mice. B cell subsets from total splenic B cell populations (singlet, live, lineage⁻, CD19⁺ gated cells) were defined as “immature-like”, based on having a lower levels of total B220 expression in 2F5 VH^{+/-} mice, relative to WT mice (left panels). The “anergic-like” phenotype is based on an accumulation of B cells in an IgM^{lo} population (in red) in 2F5 VH^{+/-} mice, distinct from any conventional splenic B cell populations (in blue), which were determined using the Kalled subfractionation scheme. This phenotype is consistent with the minimal level of serum antibody reactivity to the MPER epitope or ANA in these mice (data not shown).

FIG. 5 provides a demonstration of a subset of autoreactive+MPER epitope-reactive B cells within the 2F5 heavy chain-expressing repertoire of 2F5 V_{H} knock-in mice. The naïve repertoire of B cells in 2F5 V_{H} knock-in mice was determined by a analysis of B cell hybridomas, generated by fusions performed using NSO murine myeloma cells and spleen cells taken from unimmunized 2F5 V_{H}^{+/a} (i.e. 2F5 V_{H} IgH^{b}×WT IgH^{a}) or 2F5 V_{H}^{+/+} mice. The number of 2F5 heavy chain-expressing clones in 2F5 V_{+/a} mice was estimated by screening hybridomas using an IgM^{b}-specific ELISA assay, whereas those in 2F5 VH^{+/+} mice was estimated using a total IgM-specific ELISA assay. Note that a greater fraction of clones within the 2F5 V_{H}^{+/+} repertoire are cardiolipin and MPER reactive, relative to the 2F5 V_{H}^{+/-} repertoire, likely due to the fact that peripheral B cells from 2F5 V_{H}^{+/+} mice do not have the option of eliminating autoreactivity by usage of the endogenous heavy chain allele.

Two strategies aimed at testing MPER lead candidate immunogens in 2F5 knock-in mice in the absence of negative selection pressure are set forth in FIG. 6.

### Example 2

### Experimental Details

Expression/characterization of m2F5 and generation of 2F5 V_{H} mice. The methods and reagents used to generate m2F5 and the binding, immunofluorescence, and neutralization assays used to characterize its functional properties are described in Example 3, as are the reagents and methods used for the site-directed targeting of 2F5 V_{H} into the mouse Igh locus.

Mice and flow cytometry. Female C57BL/6 and C57BL/6 Igh^{a}, inbred mouse strains (8-12 wks of age) were purchased from Charles River Laboratories. 3H9 mice, originally produced in the laboratory of Dr. Martin Weigert on a BALB/c background, were backcrossed onto the C57BL/6 background for >14 generations in the laboratory of Dr. Robert Eisenberg (University of Pennsylvania, Philadelphia, Pa.).

For flow cytometric analysis, BM cells and splenocytes were isolated from 9-12 is week old female mice. Total BM B-cells (gated as singlet, live, CD19⁺, lin⁻ lymphocytes; lin=Ter 119, Gr-1, CD11b, CD4, CD8) were stained with APC anti-B220 and PE anti-CD43 antibodies or FITC anti-IgD and PE anti-IgM antibodies; singlet, live, lymphocyte-gated splenocytes were stained using the combination of FITC anti-B220, PE anti-IgM, APC anti-CD93, and PE-Cy7 anti-CD23 antibodies. Data were acquired using a BD LSRII flow cytometer equipped with FACS Diva software and analyzed using FloJo software.

Allotype screening. 2F5 V_{H} IgH^{b}/WT IgH^{a} and WT IgH^{b}/WT IgH^{a} F1 mice were generated by breeding C57BL/6 Igh^{a} congenic mice with 2F5 V_{H}^{+/-} mice and WT littermate controls, respectively. BM cells and splenocytes from 8-16 week old female F1 mice from each group were surface stained with PE-IgM^{a} and FITC-IgM^{b} antibodies, distinguishing targeted 2F5 V_{H} μHCs bearing the allotype of the targeted IgH allele (IgM^{b}) from endogenous μHCs bearing the IgM^{b} allotype.

ELISA analyses. Serum samples were collected from naïve female WT, 2F5 V_{H}^{+/-}, and 2F5 V_{H}^{+/+}, and where applicable, MRL/lpr mice. Serum concentrations of total IgG and IgM were determined using quantitative mouse IgG and IgM ELISA kits, respectively (Bethyl). ELISA measurements of cardiolipin and gp41 MPER 2F5 reactivity of total (IgM+IgG-specific) Igs was determined by optical density readings, as previously described (Haynes et al, Science 308:1906-1908 (2005), Alam et al, J. Immunol. 178:4424-4435 (2007)), and serum reactivity of total Igs to nuclear auto-antigens was determined using a mouse anti-ANA quantitative ELISA kit (Alpha Diagnostics). Cardiolipin and ANA assays were done using serum from 12-32 weeks; all other assays were done using serum from 8-16 week old mice.

### Results

The human 2F5 VDJ rearrangement forms functional chimeric antibodies with mouse C_{H}. An in vitro test was first made to determine whether mouse C regions impacted the association and binding properties of the original human IgG1 2F5 mAb (herein referred to as h2F5). To do this, 2F5 V_{H}/mouse Cγ₁ and 2F5 V_{L}/mouse Cκ expression constructs were generated and co-transfected into 293T cells. The 2F5 chimeric mouse/human recombinant antibody (m2F5) was assessed for its ability to bind lipid and mouse and human cell antigens. Indeed, m2F5 bound both gp41 and lipids comparably to the human IgG1 2F5 mAb (h2F5; FIGS. 8A and 8B). Moreover, m2F5, like h2F5, reacted with both human epithelial and mouse fibroblast nuclear antigens (FIGS. 8C and 8D), and neutralized HIV-1 (FIG. 8 legend and Table 1).

TABLE 1
HIV-1 neutralization activity profiles of the chimeric,
recombinant 2F5 antibody (m2F5) and human 2F5 mAb (h2F5)

IC₅₀ for neutralization of HIV-1
(μg/mL)*

| HIV-1 isolate | m2F5 | h2F5 |
|---|---|---|
| A.92UG0237 | 0.35 | 0.063 |
| B.BG1168 | 4.50 | 0.75 |
| B.SF162 | 10.23 | 0.81 |
| B.JRFL | 11.94 | 1.76 |
| B.QH0692 | 2.97 | 0.48 |
| C.TV-1 | >25 | >25 |

*50% neutralization titers (IC₅₀) as determined in the TZM-bl HIV-1 envelope pseudovirus infectivity assay.

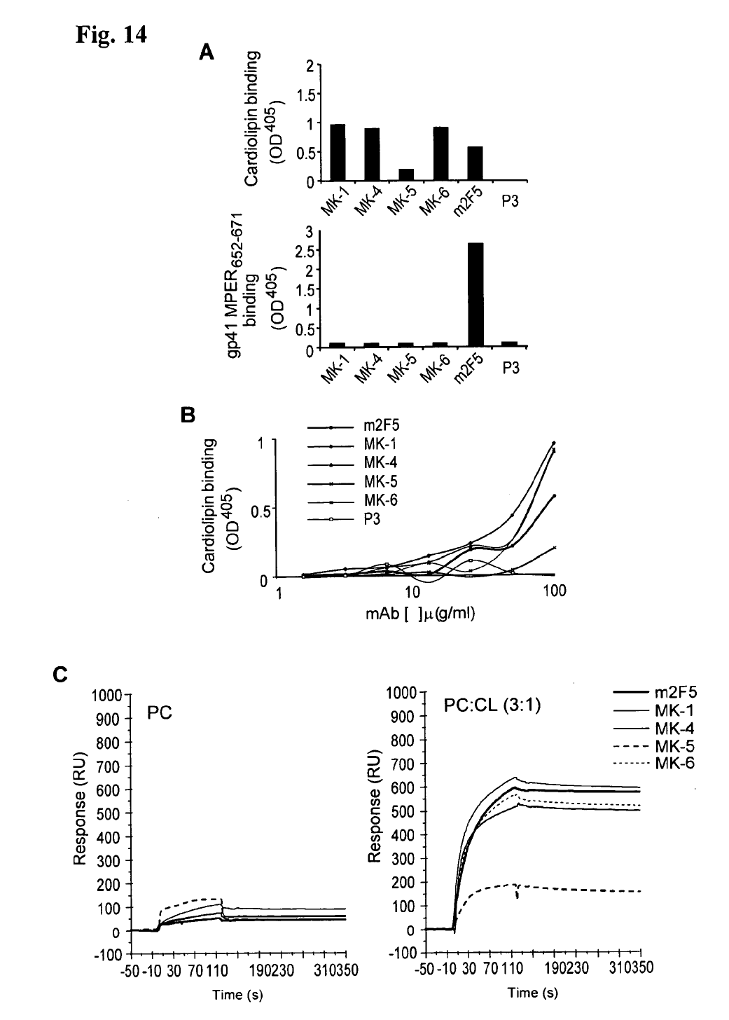

Also assessed was the ability of chimeric 2F5 HCs to pair with mouse K light chains (LC) in vitro by co-transfection of the 2F5V_{H}/mouse Cγ₁ expression construct with mouse κ LCs obtained from C57BL/6 splenic B cells by 5′ RACE PCR. To do this, four mouse κ LCs were arbitrarily selected to include the 4-52, 4-60, 4-70, and 9-96 V genes representing two Vκ families (Vκ4 and Vκ9) frequently-utilized in the splenic C57BL/6 LC repertoire. In each case, co-transfections of the m2F5 HC resulted in the production of secreted, functional mAbs (Table 2). Significantly, of the four chimeric recombinant antibodies generated by these transfections, three exhibited cardiolipin polyreactivity as determined by surface plasmon resonance and ELISA (FIG. 14).

TABLE 2
Specificities and yields of purified recombinant
mAbs from 293T cells cotransfected with the chimeric
2F5 HC and random mouse light chains

| Name of | Specificity of mouse LC | Yield of |
|---|---|---|
| recombinant mAb | cotransfected with m2F5 HC | mAb, μg/mL |
| MK-1 | Vκ4-52-Jκ4 | 0.22 |
| MK-4 | Vκ9-96-Jκ2 | 0.23 |
| MK-5 | Vκ4-60-Jκ5 | 0.34 |
| MK-6 | Vκ4-70-Jκ1 | 0.17 |
| m2F5* | m2F5 LC^{†} | 0.66 |

*Positive control produced by cotransfection of construct containing human 2F5 V(D)J rearrangement fused to mouse IgG1 and human 2F5 V(J) rearrangement fused to murine κ-chain constant region.
^{†}LC containing 2F5 V-1 rearrangement fused to mouse κLC constant region.

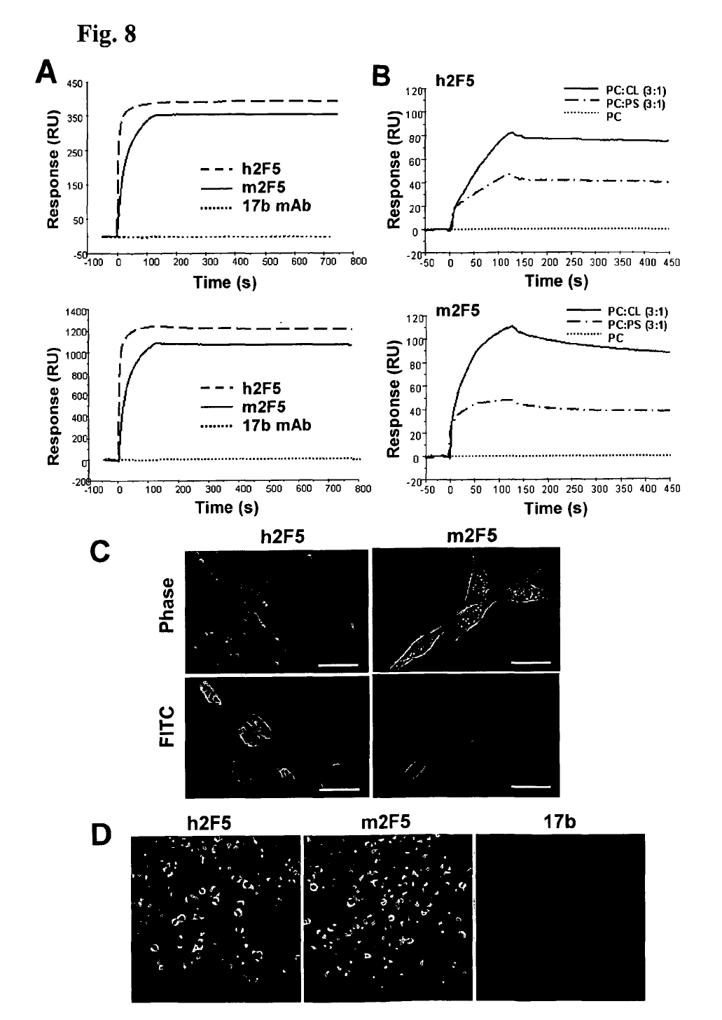

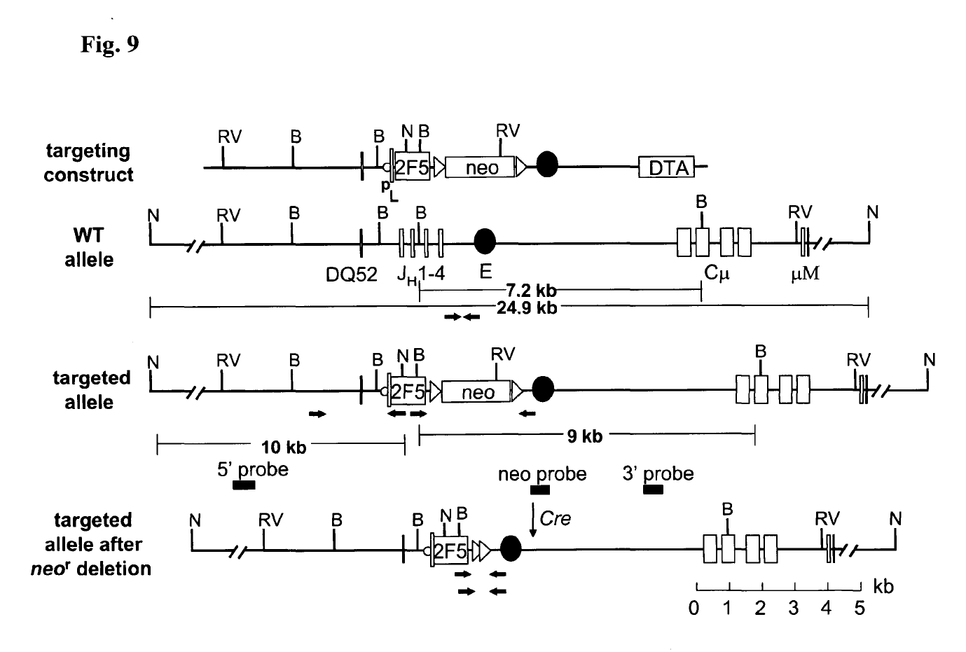

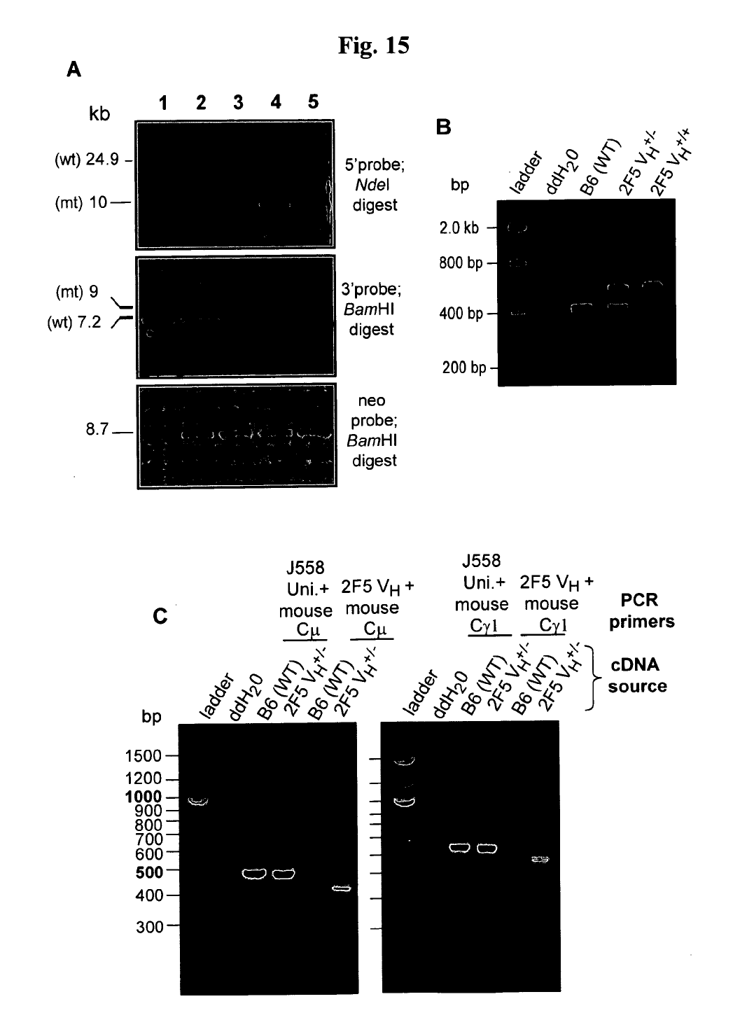

Generation of 2F5 V_{H} knock-in mice. To determine if the 2F5 mAb HC was sufficiently autoreactive to be regulated by immunological tolerance, the original, somatically-mutated 2F5 V_{H}DJ_{H} rearrangement (Muster et al, J. Virol. 68:4031-4034 (1994), Muster et al, J. Virol. 67:6642-6647 (1993)) was knocked into the mouse igh locus, replacing the J_{H}1-4 region (FIG. 9). To confirm the expected homologous recombination event in the Igh locus, four independent ES clones were assessed for the predicted insertion (FIG. 15A), and heterozygote and homozygote offspring harboring germline transmission of the 2F5 VDJ rearrangement (2F5 V_{H} knock-in mice), were identified by PCR (FIG. 15B). 2F5 V_{H} knock-in mice supported CSR to the endogenous Cγ1 locus in vivo (FIG. 15C), and thus provide a valid model for direct determination of whether the 2F5 V_{H}DJ_{H} can induce B-cell tolerance mechanisms.

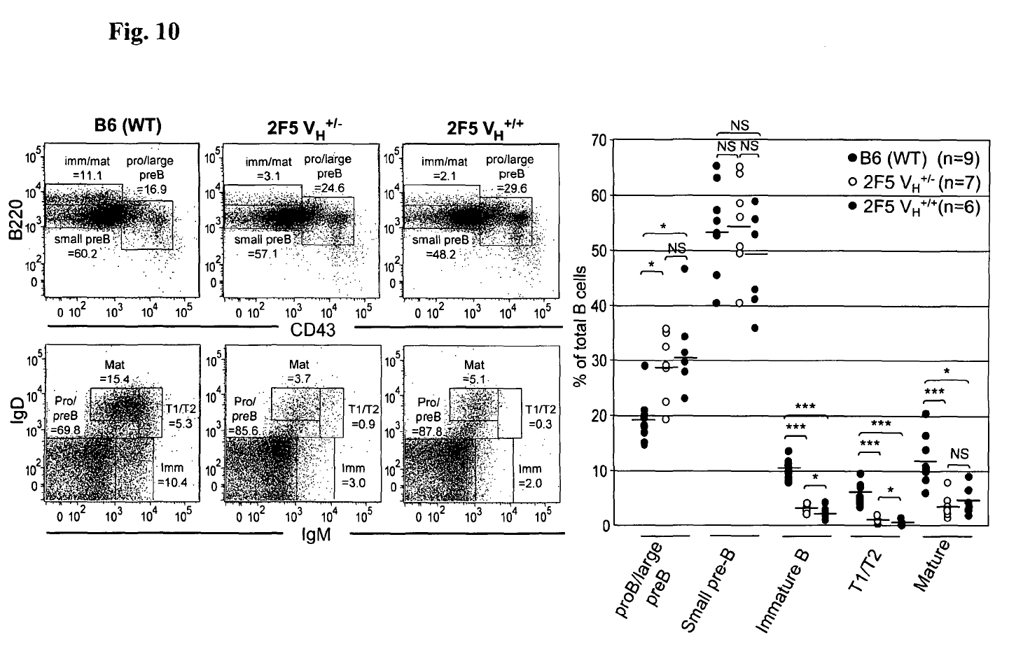

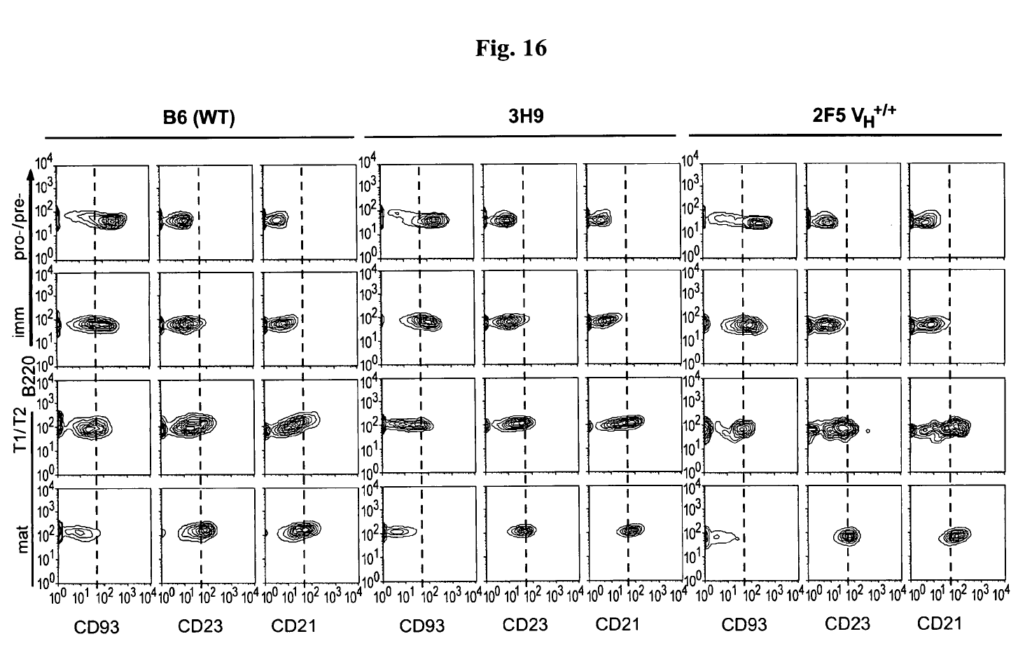

The majority of B cells expressing 2F5 V_{H} are deleted in the BM at the pre-B to immature B-cell stage. To examine the effect of the targeted 2F5 VDJ insert at one or both Igh alleles on B-cell development, a comparison was made of B-cell ontogeny in BM of heterozygous (2F5 V_{H}^{+/-}) and homozygous (2F5 V_{H}^{+/+}) knock-in mice with that of C57BL/6 controls. Fractionation of total BM B-cells from 2F5 V_{H}^{+/-} and 2F5 V_{H}^{+/+} mice into pro-B/large pre-B (B220^{lo}CD43⁺), small pre-B (B220^{lo}CD43⁻), and immature/mature B (B220^{hi}CD43⁻) fractions (Hardy et al, J. Exp. Med. 173:1213-1225 (1991)) demonstrated a profound reduction in surface immunoglobulin (sIg⁺) B-cell subsets (B220^{hi}CD43⁻), both in frequency (˜4-fold for both 2F5 V_{H}^{+/-} and 2F5 V_{H}^{+/+} mice; FIG. 10) and absolute numbers (˜10-fold for both 2F5 V_{H}^{+/-} and 2F5 V_{H}^{+/-} mice; Table 3). BM B-cells were also labeled with antibodies specific for IgM and IgD to identify immature, transitional, and mature B-cell populations. The frequency and absolute number of each population were also reduced in 2F5 V_{H} mice, with the largest decreases observed in transitional B-cell populations (˜7- or ˜20-fold reduced frequencies and ˜15- or ˜60-fold decreases in numbers in 2F5 V_{H}^{+/-} and 2F5 V_{H}^{+/+} mice, respectively). These results demonstrated that 2F5 V_{H} mice exhibited a major blockade in B-cell development predominantly at the pre-B to immature B-cell transition, which is consistent with the induction of tolerance by the deletion of immature B cells expressing the 2F5 Ig HC paired with many endogenous LCs. This developmental blockade at the immature B-cell stage is similar to that previously reported for the autoreactive anti-DNA 3H9 knock-in mouse (Chen et al, Nature 373:252-255 (1995), Sekiguchi et al, J. Immunol. 176:6879-6887 (2006)), and FIG. 16, Table 4).

TABLE 3
Cell numbers of BM B cell fractions in 2F5 V_{H} knock-in mice and WT littermate controls

| | | | B cell subset | | | | |
|---|---|---|---|---|---|---|---|
| | | | Hardy A-C′ | | | | |
| | | Total B | proB/large | Hardy D* small | Immature B | Transitional B | Mature B |
| Genotype | n | cells, ×10⁶ | preB, ×10⁵ | preB, ×10⁵ | (B220^{int}IgM^{lo}IgD⁻), ×10⁵ | (B220^{int}IgM^{int}IgD^{lo/-}), ×10⁵ | (B220^{hi}IgM^{int}IgD^{hi}), ×10⁵ |
| B6 (WT) | 8 | 5.3 ± 0.9 | 10.1 ± 2.1 | 33.9 ± 6.9 | 5.7 ± 1.4 | 2.6 ± 0.5 | 6.7 ± 1.2 |
| 2F5 V_{H}^{+/-} | 8 | 2.7 ± 0.3 | 7.0 ± 0.4 | 12.7 ± 1.9 | 0.71 ± 0.21 | 0.18 ± 0.03 | 1.0 ± 0.2 |
| 2F5 V_{H}^{+/+} | 6 | 1.9 ± 0.5 | 5.9 ± 1.6 | 9.5 ± 1.9 | 0.46 ± 0.15 | 0.04 ± 0.01 | 1.0 ± 0.3 |

TABLE 4
Comparison of BM B cell subset numbers in 2F5 V_{H} and
3H9 homozygous knock-in mice: Hardy's fractions

| | | CD43^{Hi} B220^{Lo} | | | CD43^{Lo} B220^{Hi} | |
|---|---|---|---|---|---|---|
| Mouse | Total cells | Fraction A | Fraction B | Fraction C-C′ | Fraction D | Fraction E-F |
| B6 (WT) (n = 4) | 44.1 ± 8.2 | 0.92 ± 0.32 | 0.82 ± 0.61 | 0.44 ± 0.06 | 4.68 ± 1.9 | 5.18 ± 2.4 |
| 2F5 V_{H}^{+/+} (n = 2) | 30.8 ± 8.1 | 0.99 ± 0.16 | 0.43 ± 0.13 | 0.16 ± 0.04 | 1.43 ± 0.3 | 0.38 ± 0.01 |
| 3H9 (n = 2) | 39.0 ± 2.8 | 0.9 ± 0.4 | 0.76 ± 0.13 | 0.12 ± 0.06 | 2.90 ± 0.99 | 0.98 ± 0.26 |

Values are absolute cell numbers (×10⁶ ± SD).

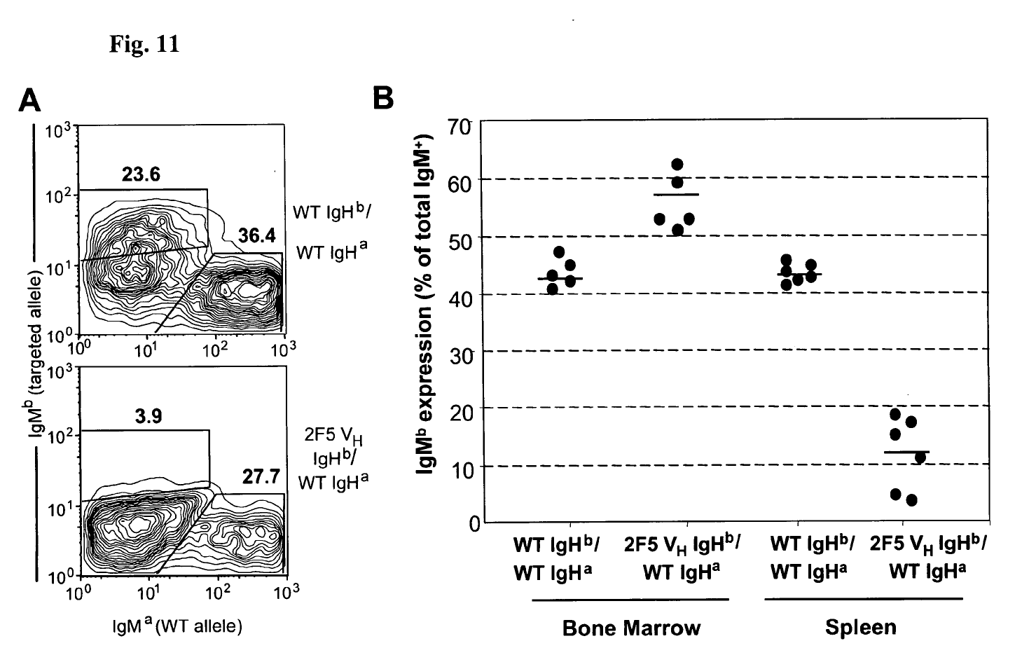

2F5 V_{H}^{+/-} knock-in splenic B cells preferentially express endogenous heavy chains. It was suspected that if 2F5 HC-expressing B-cells it heterozygote 2F5 V_{H} mice escaped BM deletion, they should be counter-selected in favor of B cells expressing endogenous Igh rearrangements, as in 3119-76R mice (Chen et al, Nature 373:252-255 (1995)). Thus, an examination was made of surface expression of endogenous (IgM^{a}) relative to 2F5 V_{H}-targeted (IgM^{b}) alleles in 2F5 V_{H}^{+/-} IgM^{a}IgM^{b} F1 (F1) mice. Indeed, most IgM⁺ splenocytes from 2F5 F1 mice expressed surface IgM^{a}, indicating strong selection for the endogenous HC (FIG. 11A). This finding contrasts with IgM⁺ B cells in the BM, where allelic exclusion of the endogenous allele is largely maintained (FIG. 11B). As measured by the relative amount of IgM^{a} and IgM^{b} expression within the total IgM+ B-cell fraction, the frequency of endogenous μHC expression in 2F5 V_{H}^{+/-} F1 mice was estimated to be ˜85% in the spleen and ˜40% in the BM (FIG. 11B). This preferential expression of the endogenous HC in splenic 2F5 V_{H}^{+/-} F1 cells suggests that 2F5 μHC-expressing peripheral B-cell populations are either selected against or eliminate their 2F5 V_{H} transgenes by intra-chromosomal recombination, followed by rearrangement/expression of the alternate allele (Chen et al, J. Immunol. 152:1970-1982 (1994)), although it cannot be formally ruled out that some of these cells may have undergone CSR. 2F5 μHC-expressing splenic B cells in 2F5 V_{H}^{+/-} F1 may have also down-modulated their B cell receptors (BCRs; FIG. 11A), a possibility that is consistent with the lower levels of IgM observed in cells that become anergized through receptor engagement (Goodnow et al, Nature 352:532-536 (1991), Goodnow et al, Nature 334:676-682 (1988)).

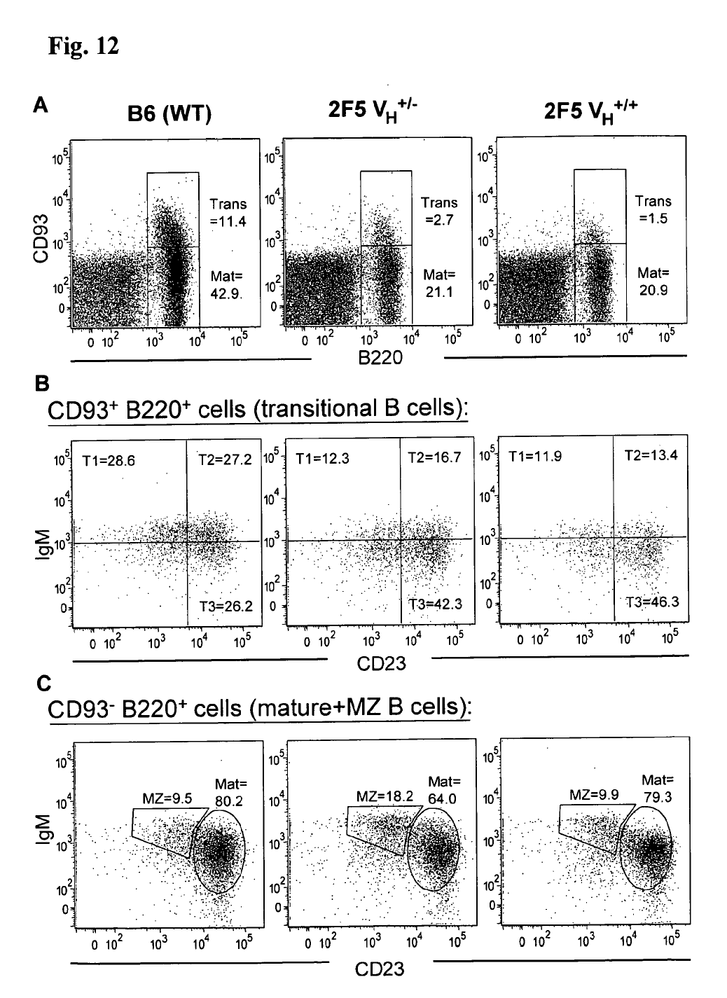

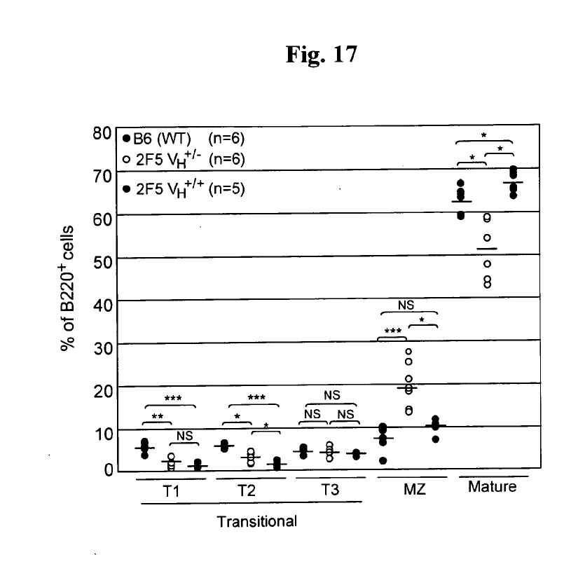

2F5 V_{H} knock-in mice have severely diminished numbers of mature splenic B-cell populations with low surface Ig density. Selection against 2F5 Ig HC⁺ BM B-cells should lead to diminished numbers of peripheral B-cells. Indeed, compared to littermate controls, the numbers of splenic B-cells (B220⁺CD19⁺lin⁻, live-gated) in 2F5 V_{H}^{+/-} and 2F5 V_{H}^{+/+} mice were reduced by 72% and 86%, respectively (Table 5). To determine if transitional, MZ, and mature B-cell subset frequencies within this remnant splenic B cell population were altered, 2F5 V_{H} B220⁺ B-cells were stained with antibodies specific for CD23, CD93, and IgM (Allman et al, J. Immunol. 167:6834-6840 (2001)). Interestingly, within this residual B cell population, the frequency of transitional IgM^{lo} (T3) B-cells was little changed, but transitional IgM^{hi} (T1 and T2) subset frequencies were significantly reduced relative to normal controls in both 2F5 V_{H}^{+/-} and 2F5 V_{H}^{+/+} mice (FIG. 12A and FIG. 17). Moreover, within the remaining total 2F5 V_{H}^{+/+} splenic B-cell population, normal frequencies of MZ B-cells and follicular mature (B220⁺, CD93⁻, CD23⁺, IgM⁺) B-cells were observed, but relative to littermate controls, the latter displayed lower surface IgM densities. This pattern of decreased T1/T2 B-cell frequencies, and relatively normal frequencies of mature B-cell subsets (but with reduced membrane Ig levels) is also quite similar to that previously reported for the 3H9 knock-in mouse (Chen et al, Nature 373:252-255 (1995), Li et al, J. Exp. Med. 195:181-188 (2002), Sekiguchi et al, J. Immunol. 176:6879-6887 (2006)). The low surface IgM densities seen in both transitional and mature B cell splenic populations of 2F5 V_{H}^{+/+} mice also mirror the low membrane IgM levels expressed by IgM^{b+} splenic B cells from 2F5 V_{H}^{+/-} F1 mice (FIG. 11A).

TABLE 5
Comparison of BM B cell subset numbers in 2F5 V_{H} and
3H9 homozygous knock-in mice: IgD vs. IgM staining

| | | | B220⁺ | | | | |
|---|---|---|---|---|---|---|---|
| Mouse | Total cells | B220⁺ | Pro/pre | Immature | T1 | T2 | Mature |
| B6 (WT) (n = 4) | 44.1 ± 8.2 | 11.4 ± 3.9 | 6.9 ± 3.5 | 1.2 ± 0.2 | 0.42 ± 0.3 | 0.44 ± 0.3 | 1.8 ± 0.5 |
| 2F5 V_{H}^{+/+} (n = 2) | 30.8 ± 8.1 | 4.1 ± 2.7 | 3.4 ± 2.2 | 0.2 ± 0.1 | 0.02 ± 0.02 | 0.01 ± 0.01 | 0.3 ± 0.2 |
| 3H9 (n = 2) | 39.0 ± 2.8 | 5.4 ± 0.1 | 4.1 ± 0.0 | 0.4 ± 0.2 | 0.08 ± 0.2 | 0.03 ± 0.02 | 0.2 ± 0.1 |

Values are absolute cell numbers (×10⁶ ± SD).

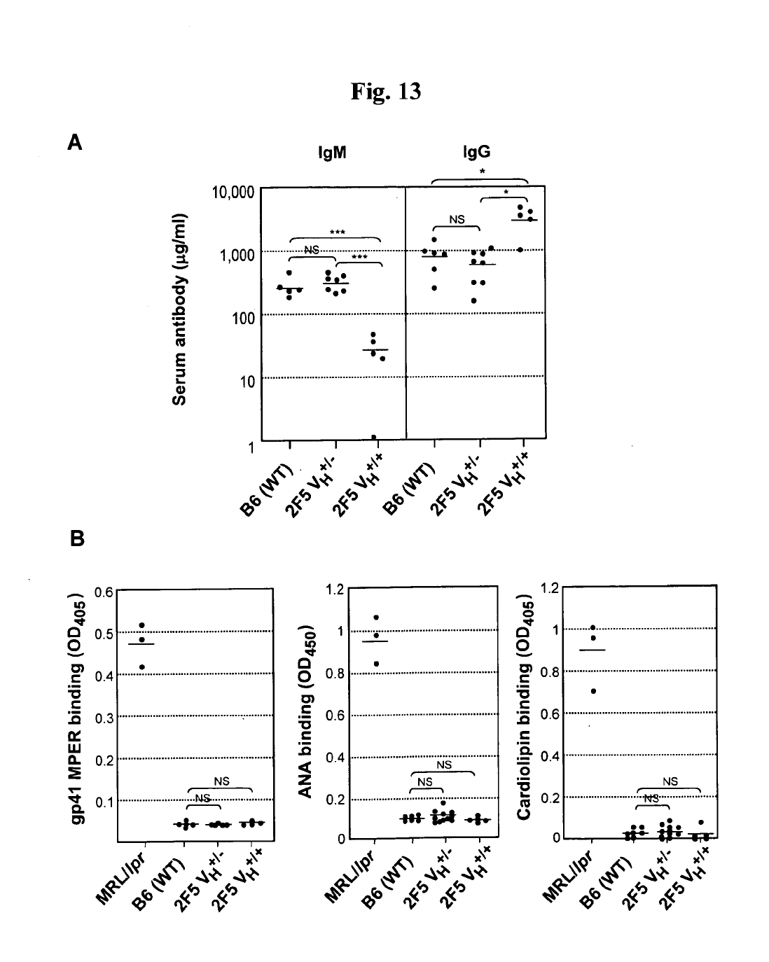

2F5 V_{H} knock-in mice lack serum reactivity to cardiolipin and anti-nuclear autoantigens despite having substantial levels of serum IgG. 2F5 V_{H}^{+/-} and 2F5 NT_{H}^{+/+} mice exhibited normal to elevated serum IgG levels relative to normal controls, respectively, but 2F5 V_{H}^{+/+} mice alone expressed significantly lower levels of serum IgM (FIG. 13A). Despite the substantial levels of circulating serum Ig in 2F5 V_{H} knock-in mice, sera from heterozygous and homozygous knock-in mice did not bind cardiolipin or nuclear autoantigens (FIG. 13B). This absence of reactivity is consistent with an autoantigen-specific blockade of B-cell development, and loss of autoreactive B-cell populations in 2F5 V_{H} knock-in mice.

In summary, the development of a safe and effective HIV-1 vaccine has been blocked by the inability to design HIV-1 immunogens that induce antibodies that potently neutralize diverse HIV-1 strains. While the HIV-1 Env has conserved regions to which rare, broadly neutralizing human antibodies bind, either on immunogens or in the context of natural infections, these conserved regions only rarely induce broadly neutralizing antibodies (Burton et al, Proc. Natl. Acad. Sci. USA 102:14943-14948 (2005), Haynes and Montefiori, Expert Rev. Vaccines 5:579-595 (2006), Simek et al, J. Virol. 83:7337-7348 (2009)). Moreover, even on the rare occasions that broadly neutralizing antibodies are induced by HIV-1 infection, they only arise months after infection (Shen et al, J. Virol. 83:3617-3625 (2009)).

That the 2F5 V_{H} knock-in mouse shows a profound block in expression of the 2F5 V_{H} at the immature B cell stage demonstrates that the 2F5 V_{H} is sufficiently autoreactive to invoke tolerance control of 2F5 V_{H} expression, and supports the notion that expression of this specificity is regulated by tolerance mechanisms in vivo. In this regard, many of the broadly neutralizing antibodies such as mAbs 4E10 and 1B12 share some characteristics of the 2F5 HC, including long hydrophobic CDR3s and polyreactivity, characteristics previously associated with antibodies marked for deletion in human BM (Meffre et al, J. Clin. Invest. 108:879-886 (2001)).

It is possible that the profound block in B cells expressing 2F5 V_{H}-containing HCs may be enhanced by incomplete and/or inefficient pairing of chimeric 2F5 V_{H}/mouse C_{H} HCs with endogenous mouse LCs. The data provided herein, however, do not support this possibility. First, the relatively normal pre-B compartment in 2F5 V_{H} knock-in mice (comparable to that in the 3H9 knock-in mouse; Tables 4-7) is most consistent with the ability of the 2F μHC to associate efficiently with surrogate LC and support continued differentiation to the immature B-cell stage, although this could also be due to compensation of an earlier, partial pre-B cell defect by autoreactive, immature B cells arrested at the pre-B cell stage. Either possibility, however, is consistent with the ability of the 2F5 μHC to form signaling-competent BCR and/or pre-BCR complexes rather than pairing incompatibility. Second, the co-transfection of the m2F5 HC with four distinct mouse LCs produced functional recombinant antibodies that reacted with self-antigens (FIG. 14). This observation demonstrates the capacity for LC pairing and the substantial penetrance of the 2F5 autoreactive phenotype. Third, 26 hybridoma lines were generated from the spleens of naïve 2F5 V_{H} knock-in mice containing the 2F5 μHC in association with κLCs utilizing 9 different Vκ gene families (Table 9). Fourth, 2F5 V_{H} knock-in mice exhibit normal ratios of MZ B cells and follicular B cells (FIG. 12) despite significant reductions in splenic B-cell numbers (Table 8). This observation indicates that the capacity for normal B-cell maturation is retained. Finally, it could be argued that the lack of serum IgM in 2F5 V_{H}^{+/+} mice is due to the 2F5 HCs inability to pair with LCs similar to the phenotype reported in κ+λ LC-chain deficient mice, which make no serum IgM, but have detectable serum IgG comprised of HC dimers (Zou et al, J. Exp. Med. 204:3271-3283 (2007)). However, 2F5 V_{H}^{+/+} knock-in mice have substantial levels of serum IgG/K as demonstrated by the use of anti-KLC Ab to capture serum IgG for ELISA quantification. Taken together, these observations strongly suggest that immunological tolerance, not impaired HC+LC pairing, is the most likely explanation for reduced B-cell numbers in 2F5 V_{H} knock-in mice.

TABLE 6
Comparison of BM B cell subset frequencies in 2F5 V_{H}
and 3H9 homozygous knock-in mice: Hardy's fractions

| | CD43^{Hf} B220^{Lo} | | | CD43^{Lo} B220^{Hi} | |
|---|---|---|---|---|---|
| Mouse | Fraction A | Fraction B | Fraction C-C′ | Fraction D | Fraction E-F |
| B6 (WT) (n = 4) | 41.9 ± 9.7 | 33.4 ± 12.1 | 21.0 ± 5.8 | 43.8 ± 15.7 | 46.6 ± 12.9 |
| 2F5 V_{H}^{+/+} (n = 2) | 58.4 ± 0.4 | 25.1 ± 3.9 | 9.8 ± 3.9 | 71.8 ± 0.8 | 19.6 ± 4.2 |
| 3H9 (n = 2) | 48.2 ± 16.8 | 42.3 ± 12.1 | 7.0 ± 4.0 | 60.9 ± 22.8 | 20.4 ± 4.7 |

Values are absolute cell frequency (% ± SD).

TABLE 7
Comparison of BM B cell subset frequencies in 2F5 V_{H} and
3H9 homozygous knock-in mice: IgD vs. IgM staining

| | | B220⁺ | | | | |
|---|---|---|---|---|---|---|
| Mouse | B220⁺ | Pro/pre | Immature | T1 | T2 | Mature |
| B6 (WT) (n = 4) | 25.2 ± 4.2 | 59.2 ± 9.1 | 11.4 ± 3.0 | 3.8 ± 1.0 | 4.2 ± 2.9 | 16.2 ± 5.7 |
| 2F5 V_{H}^{+/+} (n = 2) | 12.7 ± 5.6 | 84.0 ± 1.1 | 3.6 ± 1.0 | 0.4 ± 0.1 | 0.3 ± 0.3 | 7.3 ± 1.0 |
| 3H9 (n = 2) | 13.9 ± 0.6 | 76.4 ± 1.9 | 7.1 ± 3.7 | 1.6 ± 0.5 | 0.5 ± 0.4 | 4.6 ± 2.2 |

Values are absolute cell frequency (% ± SD).

TABLE 8
Total cell numbersin BM and spleen of 2F5 V_{H} knock-in mice and WT littermate controls

| | | BM | | | Total | Spleen | |
|---|---|---|---|---|---|---|---|
| Genotype | n | Total cells, ×10⁶ | B cells, ×10⁶ | Spleen weight, mg | cells, ×10⁶ | B cells, ×10⁶ | T cells, ×10⁶ |
| B6 (WT) | 13 | 25.3 ± 2.8^{a} | 5.5 ± 0.9^{a} | 64.6 ± 4.5^{a} | 92.7 ± 6.2^{a} | 36.1 ± 4.3^{a} | 25.5 ± 2.4^{a} |
| 2F5 V_{H}^{+/-} | 12 | 21.8 ± 1.9^{a} | 2.5 ± 0.3^{b} | 37.3 ± 3.4^{b} | 41.5 ± 3.2^{b} | 10.0 ± 1.9^{b} | 16.6 ± 1.9^{b} |
| 2F5 V_{H}^{+/+} | 6 | 22.2 ± 3.4^{a} | 1.9 ± 0.5^{b} | 29.9 ± 1.9^{b} | 26.7 ± 5.7^{c} | 5.2 ± 1.7^{c} | 11.1 ± 1.8^{c} |

Values are mean ± SD.
^{a,b,c}Groups having different letters differ at the 0.05 level, as determined by a Student's t test.

TABLE 9
Selected examples of light chain usage in
2F5 V_{H} knock-in hybridoma cell lines*

| | | Status of 2F5 V_{H} | |
|---|---|---|---|
| Hybridoma line | Source | transgene at HC | LC V family used |
| 1B1 CL2 | 2F5 V_{H}^{+/a} | + | Vκ 1-110 |
| 2A9 CL2 | 2F5 V_{H}^{+/a} | + | Vκ 8-19 |
| 2D9 CL2 | 2F5 V_{H}^{+/a} | + | Vκ 32-103 |
| 2E6 CL1 | 2F5 V_{H}^{+/a} | + | Vκ 1-117 |
| 4G7 CL1 | 2F5 V_{H}^{+/a} | + | Vκ 1-99 |
| 5B2 CL2 | 2F5 V_{H}^{+/a} | + | Vκ 4-92 |
| 1A11 CL9 | 2F5 V_{H}^{+/+} | + | Vλ1 |
| 3D3 CL4 | 2F5 V_{H}^{+/+} | + | Vκ 38-93 |
| 7C9 CL9 | 2F5 V_{H}^{+/+} | + | Vκ 4-79 |

*Hybridoma cell lines were generated by repeated subcloning of cells plated at limiting dilution from two hybridoma fusions performed using NSO murine myeloma cells and naïve spleen cells taken from either 2F5 V_{H}^{+/+}(2F5 V_{H}^{+/+}) or 2F5 V_{H} IgH^{b}/WT IgH^{a} F1 (2F5 V_{H}^{+/a}) knock-in mice. Hybridoma lines derived from 2F5 V_{H}^{+/a} mice were initially screened for 2F5-bearing HC expression by ELISA for IgH^{b}-specific antibodies in supernatants. The presence of the 2F5 V_{H}DJ_{H} insertion was verified by PCR amplification of cDNA using a primer combination specific for the 2F5 V_{H} transgene and Cμ and subsequent direct sequencing; V_{κ} families were determined by PCR amplification of cDNA with a degenerate Vκ-specific and C_{κ} primer combination, and direct sequencing. All hybridomas from this panel were of the IgM isotype and none had any mutations in the 2F5 V_{H} transgene.

The peripheral phenotype in 2F5 V_{H}^{+/+} mice is consistent with additional mechanisms for controlling autoreactivity in residual splenic 2F5 V_{H}-bearing B cells that have escaped central tolerance. In particular, the relative enrichment for the T3 IgM^{lo} population in 2F5 V_{H}^{+/+} mice (FIG. 12) is consistent with increased frequencies of autoreactive B cells that become anergized through receptor engagement (Goodnow et al, Nature 352:532-536 (1991), Goodnow et al, Nature 334:676-682 (1988)). A similar IgM^{lo} phenotype has also been described in splenic transitional B cells in the various 3H9 mouse lines (Chen et al, Nature 373:252-255 (1995), Erikson et al, Nature 349:331-334 (1991), Li et al, J. Exp. Med. 195:181-188 (2002), Chen et al, J. Immunol. 176:5183-5190 (2006), Sekiguchi et al, J. Immunol. 176:1213-1225 (1991)), reflecting frequent anergic B cells, or alternatively, cells that have undergone LC editing (Sekiguchi et al, J. Immunol. 176:1213-1225 (1991), Kiefer et al, J. Immunol. 180:6094-6106 (2008), Kakajima et al, J. Immunol. 182:3583-3596 (2009))). Interestingly, 2F5 V_{H}^{+/+} mature B cells also exhibit lower sIgM densities, similar to anergic anti-Sm transgenic mature B-cell fractions (Culton et al, J. Immunol. 176:790-802 (2006)). An intriguing alternative explanation for reduced sIgM expression in mature 2F5 V_{H}^{+/+} B-cell populations is that their autoreactive BCRs bind to an intracellular antigen, analogous to mature B cell populations in the When egg lysozyme (HEL) model (Ferry et al, J. Exp. Med. 198:1415-1425 (2003)). Regardless of the reason for the lowered IgM levels in mature 2F5 V_{H}^{+/+} B-cells, the fact that such populations are present at normal ratios, coupled with the abundance of non-autoreactive serum Igs in 2F5 V_{H}^{+/+} mice, predicts that additional mechanisms (other than anergy) purge autoreactivity in these populations. In various 3H9 knock-in lines, such additional mechanisms of tolerizing 3H9-bearing dsDNA-reactive mature B-cells include LC receptor editing, or replacement of the 3H9 insert by a secondary V→VDJ rearrangement, i.e., V_{H} replacement (Chen et al, Immunity 6:97-105 (1997), Li et al, Immunity 15:947-957 (2001), Chen et al, Immunity 3:747-755 (1995)). It will be critical to determine which of these peripheral B-cell tolerance mechanisms, and/or allergy operate in 2F5 V_{H}^{+/+} mice.

Mice bearing conventional or targeted autoreactive Ig transgenes have been critical in defining the developmental stages in which self-reactive B cells are eliminated (Shlomchik, Immunity 28:18-28 (2008)). The 2F5 VDJ knock-in mouse line demonstrates that the great majority of B-lineage cells that express the 2F5 VDJ rearrangement are halted in their development at the transition from small pre-B to immature B-cells (FIG. 10). This developmental blockade is nearly identical to that observed in mice that express the 3H9-76R VDJ rearrangement that specifies anti-DNA reactivity in association with many LCs (Li et al, Immunity 15:947-957 (2001)). If the germ-line 2F5 VDJ rearrangement does not specify autoreactivity, immature B cells carrying the 2F5 HC would not be tolerized and the 2F5 HC CDR residues critical for self-reactivity and HIV-1 neutralization must have arisen in germinal centers. Significantly, removal of autoreactive B cells can also occur in germinal centers (Han et al, J. Exp. Med. 182:1635-1644 (1995)), and it is possible that 2F5 HC B cells carrying somatically-generated mutations critical for self-reactivity/HIV-1 neutralization may normally be deleted or modified during the germinal center reaction. Future studies to determine if tolerance mechanisms act similarly on the 2F5 germline VDJ sequence will be informative and complementary to these studies.

Both the 2F5 and 4E10 mAbs bind to the gp41 membrane proximal region on HIV-1 virions as well as to the lipid bilayer (Alain et al, J. Immunol. 178:4424-4435 (2007)). Mutation of hydrophobic residues in the 2F5 HC CDR3 abrogates both lipid binding and neutralization of HIV-1 (Alam et al, Proc. Natl. Acad. Sci. USA epub (Nov. 11, 2009)). The induction of neutralizing antibodies specific for this region will likely require the targeting of B-cell populations that can make antibodies that bind both lipids and gp41 Env epitopes. This requirement may be facilitated by the activation of dendritic cells or other antigen presenting cells capable of promoting vaccine-induced B-cell responses that normally do not occur. The 2F5 mAb has been safely administered to a number of humans and 2F5 does not have characteristics of a pathogenic lipid autoantibody (i.e., it does not require (3-2-glycoprotein-1 to bind to lipids) (haynes et al, Science 308:1906-1908 (2005), de Groot and Derksen, Thromb. Haemost 3:1854-1860 (2005), Vcelar et al, AIDS 21:2161-2170 (2007)). However, if these antibodies can be induced, safety monitoring in non-human primate trials will be of paramount importance.

These studies demonstrate that the HIV-1 broadly neutralizing antibody 2F5-containing MC is sufficiently autoreactive to trigger immunological tolerance in the setting of a knock-in mouse. These findings have important implications for the design of strategies to induce neutralizing antibodies to the HIV-1 Env gp41 membrane proximal region. HIV-1 vaccine development should focus on vaccine regimens that might safely circumvent these tolerance controls. Moreover, efforts should concentrate on accelerating and broadening those neutralizing antibody responses that are readily made in response to HIV-1, such as autologous neutralizing antibodies that arise months after natural HIV-1 infection (Richnab et akm Uric, Batk, /acad, /scu, YSA 100:4144-4149 (2003), Wei et al, Nature 422:307-312 (2003)).

### Example 3

Generation and characterization of m2F5. To make m2F5, human V+mouse C constructs in which the original 2F5 V_{H} region, ligated to mouse Cγ1 (m2F5 HC) and the original 2F5 Vκ region, fused to mouse Cκ (m2F5 LC), were cloned into the pcDNA 3.1 expression vector, co-expressed in 293T cells by transient transfection, and the resulting recombinant antibody (m2F5) was purified by standard methods. For SPR binding measurements, biotinylated MPER peptides were anchored to streptavidin sensor chip and non-specific binding to scrambled version of the MPER peptide was subtracted. Liposomes with the indicated phospholipid compositions were prepared and 500 RU of each liposomes was anchored to the L1 sensor chip as described earlier (Alam et al, J. Immunol. 178:4424-4435 (2007)). Each antibody was injected at 100 μg/mL and non-specific binding of antibodies was assessed on liposomes with phosphatidylcholine. For detection of m2F5 and h2F5 reactivity with mouse nuclear antigens, NIH-3T3 cells were grown under standard conditions on glass slides and subsequently fixed and permeabilized (Wardernann et al, Science 301:1374-1377 (2003)). Fixed cells were then incubated in medium containing 100 μg/ml m2F5 or h2F5, and bound antibodies were visualized with goat anti-mouse Igκ or goat anti-human IgG-FITC, respectively, using a Zeiss Axiovert 200M confocal immunofluorescence microscope (50 ms exposure). For detection of m2F5 and h2F5 reactivity with human nuclear antigens, HEp-2 epithelial cell slides (Zeus scientific, Raritan, N.J.) were incubated with 100 μg/ml of m2F5 and h2F5 antibodies, followed by saturating amounts of goat anti-mouse Igκ (or goat anti-human Ig, and visualized as above. Human mAb 17b, a non-autoreactive CD4i gp120 mAb, was used as a negative control for background staining. The chimeric m2F5 antibody and the h2F5 antibody were tested in the standard TZMB/L pseudovirus infection inhibition assay with the Env HIV-1 pseudoviruses B.BG1168, B.SF162, B.QH0692, A.92UG037, and C.TV-1.

Generation of 2F5 V_{H} Mice: The targeting vector contained the rearranged 2F5 V_{H} gene inserted within the joining (J_{H}) region of the immunoglobulin heavy chain, disrupting all endogenous J_{H} segments. The murine immunoglobulin J_{H} region and the upstream and downstream regions from J_{H}, used to generate the 3′ and 5′ homology mills, were isolated from a mouse C57BL/6 genomic library-derived BAC clone. The targeting backbone contained CAG-DTA and loxP-flanked Neo selection cassettes. Homologous recombination of ES cells was confirmed by Southern blotting using Nde I or Bam HI. Targeted ES clones were subjected to in vitro Crc recombinase-mediated deletion of the neo selection cassette and four correctly targeted, neo⁻ clones were injected into C57BL/6J Tyr^{c-2J} blastocysts, two of which produced chimeric mice that transmitted the 2F5 V_{H} insertion. 2F5 V_{H}^{+/-} and 2F5 V_{H}^{+/+} genotypes were determined in the offspring by PCR primers specific for WT or targeted alleles and a primer common to both alleles (see FIG. 8 for vector targeting scheme and screening strategy). To detect Ig HC transcripts in 2F5 V_{H}^{+/-} or control C57BL/6 mice, 2F5 V₁ and either murine Cμ or Cγ1 specific primers were used for PCR amplification using cDNA from purified splenic B-cells. Control endogenous V_{H}1 rearrangements were detected using a primer recognizing multiple V_{H} J558 leader sequences in combination with the Cμ or Cγ1-specific primers.

### Example 4

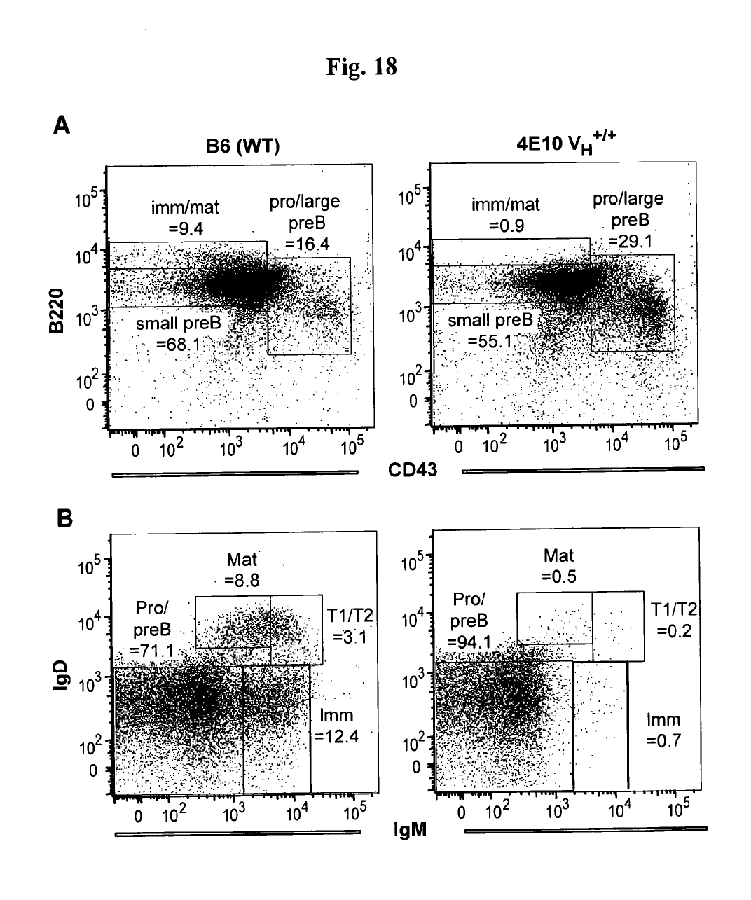

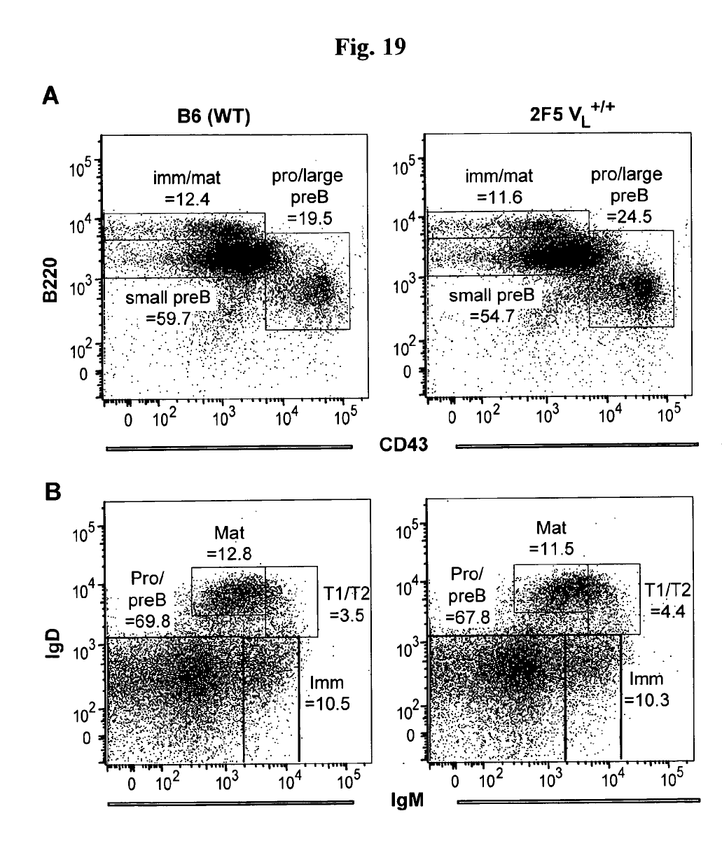

FIGS. 18 and 19 represent initial characterization (i.e., phenotypic profiles of bone marrow B cells) from two additional HIV-1 MPER knock-in strains: 4E10 VH+/+ knock-in mice (engineered to express the heavy chain of the MPER bnAb 4E10 at both alleles), and 2F5 VL+/+ knock-in mice (engineered to express the light chain of the MPER+bnAb 2F5 at both alleles).

FIG. 18 shows that the heavy chain from another MPER bnAb, 4E10 (which exhibits a similar autoreactive profile, i.e., long hydrophobic CDR3s and in vitro reactivity to self-antigens), like the 2F5 HC, is also sufficiently autoreactive to trigger tolerance in vivo. This indicates that studies performed in MPER bnAb knock-in models is generalizable, both to understanding how this class of HIV-1 bnAbs, i.e., those against this very conserved vaccine target, the MPER, are handled by tolerance mechanisms in viva, and as a readout for assessing how MPER-specific Ab responses elicited by different vaccination strategies are influenced by such mechanisms.

FIG. 19 is significant as a specificity control for two reasons. First, it shows targeted insertion of an expression construct bearing a portion of a human Ig does not have a general, non-specific effect on B cell development. Secondly, it confirms previous observations in knock-in models of well-characterized high affinity autoantibodies, in which it has been demonstrated that their light chains do not specify autoreactivity when paired with the endogenous HC repertoire, and importantly, have no accompanying effect on B cell development (this contrasts the dominant role of heavy chains of such high affinity autoantibodies in specifying their autoreactivty).

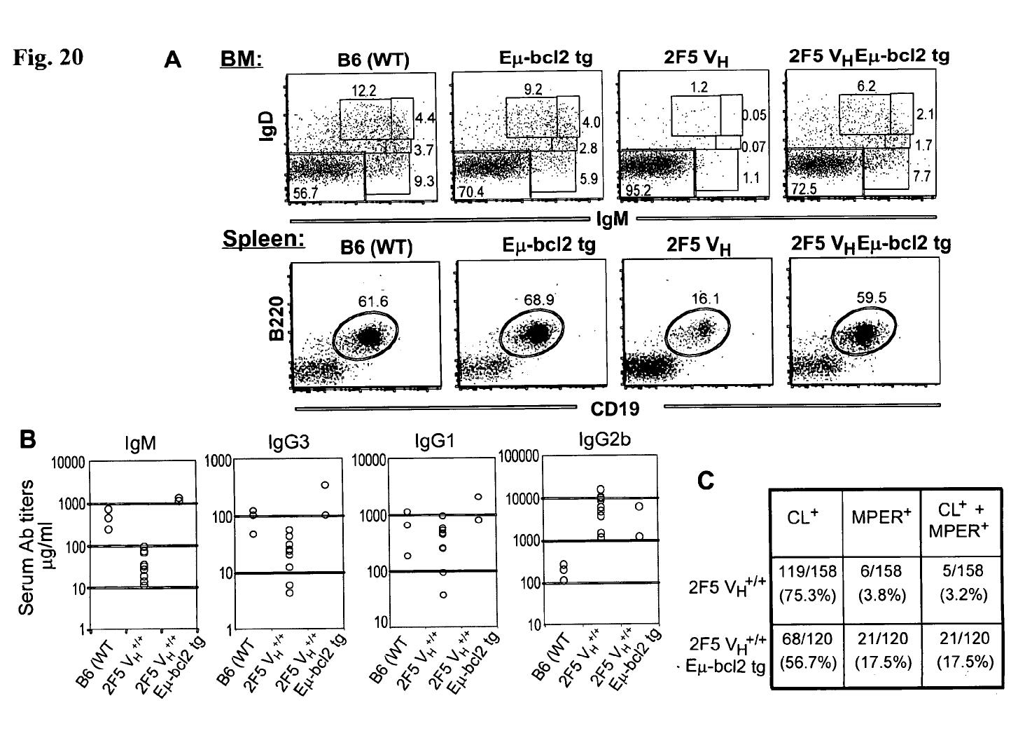

FIG. 20 represents initial data obtained from the 2F5 VH Em-bcl2 tg mouse, which was made by crossing the 2F5 VH+/+ knock-in strain with Em-bcl2 tg mice, a strain that was proposed in FIG. 6. The significance of FIG. 20 is two-fold. First, it provides additional evidence that 2F5-expressing B cells are under deletional controls, and it indicates that MPER-reactive B cells, which represent those normally extremely rare potential bnAb-producing B cell precursors, can be enriched when such deletional controls are genetically removed (FIG. 20C). Secondly, the rescue of total 2F5 VH-expressing B cell numbers in spleen and BM of 2F5 VH×bcl2 tg mice (FIG. 20A), as well the rescue of total serum IgM and IgG3 (FIG. 20B), means that 2F5 VH mice (having deletional/anergy controls) and 2F5 VH×bcl2 tg mice (without these deletional/anergy controls) can serve as elegant comparative readout models for studying how MPER Ab responses elicited by a particular immunization strategy are influenced by deletion/allergy tolerance mechanisms.

All documents and other information sources cited above are hereby incorporated in their entirety by reference.

What is claimed is:

1. A targeted transgenic mouse, the genome of said mouse comprising a nucleic acid sequence encoding a heavy or a light chain variable region of a human HIV-1 broadly neutralizing antibody, wherein said nucleic acid sequence is present in said genome operably linked to a promoter so that said nucleic acid sequence is expressed and said heavy or said light chain variable region of said human HIV-1 broadly neutralizing antibody is produced.

2. The mouse according to claim 1 wherein said human HIV-1 in broadly neutralizing antibody is 2F5.

3. The mouse according to claim 1 wherein said human HIV-1 broadly neutralizing antibody is 4E10.

4. The mouse according to claim 1 wherein said nucleic acid sequence encodes the heavy chain variable region of said human HIV-1 broadly neutralizing antibody.

5. The mouse according to claim 1 wherein said nucleic acid sequence encodes the light chain variable region of said human HIV-1 broadly neutralizing antibody.

6. The mouse according to claim 4 wherein said nucleic acid sequence encoding said heavy chain variable region of said human HIV-1 broadly neutralizing antibody is operably liked to a J558 H10 family VH promoter.

7. The mouse according to claim 5 wherein said nucleic acid sequence encoding said light chain variable region of said human HIV-1 broadly neutralizing antibody is operably liked to a VkOx-1 family Vkappa promoter.

8. The mouse according to claim 1 wherein said nucleic acid sequence is present in said genome operably linked to an endogenous enhancer element.

9. The mouse according to claim 1 wherein said genome of said mouse comprises a nucleic acid sequence encoding the heavy chain variable region of said human HIV-1 broadly neutralizing antibody and a nucleic acid sequence encoding the light chain variable region of said human HIV-1 broadly neutralizing antibody.

10. A chimeric HIV-1 broadly neutralizing antibody isolatable from said mouse according to claim 1.

11. A chimeric HIV-1 broadly neutralizing antibody isolatable from said mouse according to claim 9.

12. The chimeric antibody according to claim 11 wherein said chimeric antibody comprises the heavy and light chain variable regions of 2F5.

13. The chimeric antibody according to claim 11 wherein said chimeric antibody comprises the heavy and light chain variable regions of 4E10.

14. A hybridoma derived by fusing antibody-producing B cells of said mouse according to claim 1 with myeloma cells.

15. Monoclonal antibodies produced by the hybridoma of claim 14.

16. A method of identifying a candidate agent capable of inducing the production of HIV-1 broadly neutralizing antibodies comprising:
i) administering to said mouse according to claim 1 a test compound under conditions such that antibodies can be produced or such that B cells can be induced to express antibodies,
ii) obtaining an antibody-containing sample or an antibody expressing, B cell-containing sample from said mouse, and
iii) assaying said sample for the presence or absence of antibodies specific for the HIV-1 membrane proximal external region (MPER), or MPER-specific B cells, wherein the presence of said MPER-specific antibodies or B cells in said sample, relative to a control sample, indicates that said compound is said candidate agent.

17. The method according to claim 16 wherein said antibody-containing sample or said antibody expressing, B cell-containing sample is a serum sample or a sample of a mucosal extract.

18. The method according to claim 17 wherein said mucosal extract sample is a saliva, stool or vaginal wash sample.

19. The method according to claim 17 wherein said B-cell containing sample is a sample obtained from a systemic or mucosal immune tissue of said mouse.

20. The method according to claim 19 wherein said sample is a bone marrow, spleen or peripheral blood lymphocyte sample.

21. The method according to claim 19 wherein said sample is an enteric lymph node or Peyer's patch sample or female reproductive tract sample or lung sample.

22. The method according to claim 16 wherein step (iii) is effected using an ELISA, ELISPOT, Surface Plasmon Resonance, Lurninex or flow cytometry-based assay.

23. The method according to claim 16 further comprising assaying said candidate agent for HIV-1 neutralizing activity.

24. The method according to claim 23 wherein said agent is assayed for neutralizing activity using a TZM-bl assay.

25. The method according to claim 16 wherein said test compound comprises a protein or peptide.

26. A method of identifying an agent capable of inducing the production of HIV-1 broadly neutralizing antibodies comprising:
i) administering to said mouse according to claim 1 a test compound under conditions such that antibodies can be produced or such that B cells can be induced to express antibodies,
ii) obtaining an antibody-containing sample or an antibody expressing, B cell-containing sample from said mouse, and
iii) assaying said sample for HIV-1 neutralizing activity, relative to a control sample.

27. The method according to claim 25 wherein step (iii) is effected using a TZM-bl assay.
